(19) United States
(12) Patent Application Publication
(10) Pub. No.: US 2025/0336483 A1
(43) Pub. Date: Oct. 30, 2025

(54) RAPID ESTIMATION OF INTERMOLECULAR ELECTRONIC COUPLING AND CHARGE-CARRIER MOBILITY OF ORGANIC MOLECULES THROUGH A MACHINE-LEARNING PIPELINE

(71) Applicant: University of Kentucky Research Foundation, Lexington, KY (US)

(72) Inventors: Vinayak Bhat, Lexington, KY (US); Chad Risko, Lexington, KY (US)

(21) Appl. No.: 19/193,548

(22) Filed: Apr. 29, 2025

Related U.S. Application Data
(60) Provisional application No. 63/640,174, filed on Apr. 29, 2024.

(51) Int. Cl. G16C 10/00; G16C 20/70

(52) U.S. Cl. CPC G16C 10/00; G16C 20/70

(57) ABSTRACT

This disclosure is directed to machine learning-based methods for modelling the intermolecular electronic couplings of organic molecules. The method comprises inputting synthetically generated graph representations of at least two organic molecules into a machine learning system. The machine learning system predicts an intermolecular coupling property (V) between the at least two organic molecules from the molecular graph representations. The machine learning system further determines an anisotropic charge-carrier mobility value for the organic molecule from the predicted intermolecular coupling property. The machine learning system then determines whether the anisotropic charge-carrier mobility value meets or exceeds a predetermined threshold anisotropic charge-carrier mobility value. Machine learning systems for modelling intermolecular electronic couplings of at least two organic molecules, and methods for training the machine learning systems, are described also.

### RELATED APPLICATIONS

This application claims priority to U.S. provisional patent application Ser. No. 63/640,174 filed on Apr. 29, 2024, the entirety of the disclosure of which is incorporated herein by reference.

### GOVERNMENT INTEREST

This invention was made with partial government support under award numbers DMR-1627428 and TG-CHE200119 awarded by the National Science Foundation, and award number N00014-19-12453 from the Office of Naval Research. The government has certain rights in the invention.

### TECHNICAL FIELD

The presently disclosed subject matter generally relates to machine learning methods for analyzing organic semiconductor properties. In particular, the disclosure relates to machine learning methods and systems for modelling intermolecular electronic coupling properties between organic molecules, suitable in applications such as optoelectronic applications. The machine learning methods and systems find utility in a variety of applications, including without intending any limitation optoelectronic applications such as predicting the suitability of organic molecules in organic molecule-based semiconductors.

### BACKGROUND

Organic semiconductors (OSC) offer tremendous potential across a wide range of (opto)electronic applications. OSC development, however, is often limited by trial-and-error design, with computational modeling approaches deployed to evaluate and screen candidates through a suite of molecular and materials descriptors that generally require hours to days of computational time to accumulate. Such bottlenecks slow the pace and limit the exploration of the vast chemical space comprising OSC.

Intermolecular electronic couplings (or transfer integrals) in organic semiconductors (OSC) are critical parameters governing charge-carrier transport.¹⁻⁵ The intermolecular electronic couplings depend both on the geometric overlap of neighboring molecules (and, hence, intermolecular vibrational or phonon modes) and the molecular orbital (MO) overlap of these adjacent molecules—e.g., between the highest-occupied molecular orbitals (HOMO) of the two molecules (HOMO-HOMO coupling) for hole transport, or the lowest-unoccupied molecular orbitals (LUMO) of the two molecules (LUMO-LUMO coupling) for electron transport.^{3,6} The phases of the intermolecular electronic couplings are determined by the MO overlap symmetries.

Several approaches have been implemented to determine intermolecular electronic couplings.^{4, 7-11} In the energy-splitting-in dimer method,^{8,12} the intermolecular electronic coupling is estimated to be one-half the energy difference between the HOMO and HOMO-1 of a (noncovalent) dimer formed by two adjacent molecules (see FIG. 1 for representation of a molecular dimer geometry). While this method is effective for symmetrically arranged molecules, the method fails for systems where molecular asymmetry leads to polarization. This shortcoming is overcome in the fragment molecular orbital (FMO) approach, wherein an orthonormal basis is used to preserve the local character of the monomer orbitals.^{10, 13, 14} Via the FMO approach, the effective intermolecular electronic coupling (V₁₂) between the adjacent molecules (denoted by the numbers 1 and 2) in a molecular dimer is given by

$${V}_{12} = \frac{{H}_{12} - 0.5 \times {S}_{I 2} \times ( {H}_{1} + {H}_{2} )}{1 - {S}_{12}^{2}} \qquad ( 1 )$$

where H₁₂ is the interaction energy or electronic coupling matrix, S₁₂ is the overlap integral, and H₁ and H₂ are the monomer site energies. While there are extensions of the FMO approach beyond molecular dimers to include many-body interactions,¹⁵ band dispersion interactions in crystalline organic systems are typically weak (0.1-0.5 eV), and restricting evaluations to molecular dimers yields reasonable estimates of the intermolecular electronic couplings.¹⁶ Application of the FMO approach typically makes use of wavefunction or density functional theory (DFT) calculations, which can afford high accuracy, though they can be time-consuming; semi-empirical wavefunction methods do offer faster evaluations, often at the cost of accuracy.

With determinations of the intermolecular electronic couplings in hand, OSC charge-carrier transport characteristics can be evaluated by including these descriptors with kinetic Monte Carlo methods,¹⁷⁻¹⁹ molecular dynamics (MD) simulations,^{20, 21} or transient localization theory.^{22, 23} However, each of these approaches requires that a large number of intermolecular electronic couplings be evaluated with high accuracy and, ideally, limited computational cost. Recent efforts have sought to develop fast yet reliable machine learning (ML) models to predict intermolecular electronic couplings.²⁴⁻³³ The underlying idea of training an ML model is to acquire accurate, near-real-time predictions of desired properties (also called fast online performance) while amortizing the cost via an expensive offline dataset creation, curation, and model training campaign. For intermolecular electronic couplings, a key step is that molecular dimer geometries must be transformed to ML model input. One of the commonly used transformations is the coulomb matrix,³⁴ wherein the matrix element between two atoms i and j is defined by:

$${C}_{ij} = \{ \begin{matrix}0.5 {Z}_{i}^{2.4} & ∀ i = j \\ \frac{{Z}_{i} {Z}_{j}}{‖ {R}_{i} - {R}_{j} ‖} & ∀ i \neq j\end{matrix} \qquad ( 2 )$$

where Zᵢ and Zⱼ represent the nuclear charges of the atoms, Rᵢ and Rⱼ are the atomic Cartesian position vectors, and ∥Rᵢ−Rⱼ∥ is the Euclidean distance between atom i and atom j. Here, the size of the matrix depends on the number of atoms in the molecule. Gagliardi and coworkers used the coulomb matrix representation for dimer geometries as input for a kernel ridge regression (KRR)-based ML model²⁸ that was trained on a dataset that contained dimer geometries extracted from an MD simulation of a pentacene crystal. This work demonstrated that an ML model could be trained to yield intermolecular electronic couplings for a pentacene crystal with low prediction error. Wang et al. adopted a similar approach and used 250,000 naphthalene dimers extracted from MD simulations to train a KRR and artificial neural network (ANN) model with a coulomb matrix.²⁷ ML models trained by this approach, however, are generally not transferable, as coulomb matrix sizes vary for different molecules; hence, one needs to generate data and train models for each new system to be investigated.^{27, 35} The redundancy of training one model for every molecule can be reduced by training a single model with a fixed size coulomb matrix representation for all data, as demonstrated by Reiser et al.;²⁶ here, the size of the coulomb matrix representation for molecular dimers is fixed according to the largest molecule in the dataset, and the representation padded with zeros for smaller molecules to maintain the fixed size. Though this representation yields a transferable ML model for systems with a similar number of atoms, the large variation in atom numbers leads to sparsity in the representation, resulting in limited model performance.³⁵

An alternative to the coulomb representation is the graph representation, where atoms correspond to nodes of the graph and bonds are the edges.³⁶ For example, if one considers benzene to be represented as a graph, the carbon and hydrogen atoms are represented by nodes, and the bonds between the atoms are represented as edges; node features include atom type and hybridization state, while edge features include bond type and length. We demonstrated in previous work that the graph representation coupled with a message-passing neural network (MPNN) can be used to predict electronic, redox, and optical properties of organic π-conjugated molecules with DFT-level accuracy.³⁷ In MPNN, information from neighboring nodes is aggregated and processed at each node. This allows the ML model to learn how the local environment influences each atom. Unlike the coulomb matrix, the graph representation does not depend on the number of atoms in the system; hence, the graph representation offers a more transferable approach compared to the coulomb matrix. Notably, graph representations have been previously proposed to predict intermolecular electronic couplings.³⁵

When considering charge-carrier transport in OSC, a key parameter of interest is the intermolecular electronic coupling. Here, we introduce a machine learning (ML) model to predict intermolecular electronic couplings in organic crystalline materials from their three-dimensional (3D) molecular geometries. The ML predictions take only a few seconds of computing time compared to hours by density functional theory (DFT) methods. To demonstrate the utility of the ML predictions, we deploy the ML model in conjunction with mathematical formulations to rapidly screen the charge-carrier mobility anisotropy for more than 60,000 molecular crystal structures and compare the ML predictions to DFT benchmarks.

In this work, we use graph representations to predict intermolecular electronic couplings from molecular dimer geometry using SphereNet, a graph-based three-dimensional (3D) MPNN.³⁸ For a 3D MPNN, the input representation includes 3D coordinates of each atom written in the graph format described above, thereby capturing the molecular spatial arrangements, a crucial feature for predicting properties dependent on molecular shape/structure and intermolecular interactions. SphereNet has been used to predict molecular properties such as the dipole moment, polarizability, and free energy from a 3D molecular geometry.³⁸ The input for training SphereNet used here includes the atomic Cartesian coordinates (x, y, z) of molecules in a dimer and corresponding atomic numbers (Z), as shown in FIG. 1. These coordinates are then transformed into a 3D graph representation using spherical coordinates (d, 0, 4). For more in-depth information on SphereNet, see Liu et al.³⁸ We demonstrate that the SphereNet architecture, when trained with a diverse dataset of 438,000 DFT-derived intermolecular electronic couplings from over 25,000 molecular crystal structures in the OCELOT (Organic Crystals in Electronic and Light-Oriented Technologies) database,³⁹ provides a highly transferable ML model. Furthermore, we develop and deploy an open-access ML pipeline that uses the predicted intermolecular electronic couplings to estimate charge-carrier mobility anisotropies within the semi-classical Marcus theory approach proposed by Goddard and coworkers;⁴¹ the reorganization energy parameters used to derive the Marcus theory hopping are also predicted via a pre-trained ML model.³⁷ Using this ML pipeline, we are able to rapidly screen vast numbers of molecular organic crystals for their capacity to transport charge carriers.

### SUMMARY

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

In one aspect, the present disclosure is directed to a machine learning-based method for modelling the intermolecular electronic couplings of organic molecules. The method comprises inputting synthetically generated graph representations of at least two organic molecules into a machine learning system. The graph representations are molecular graph representations in which each node corresponds to an atom of the organic molecules and each edge corresponds to a chemical bond between atoms of the organic molecules. The machine learning system predicts an intermolecular coupling property (V) between the at least two organic molecules from the molecular graph representations. The machine learning system further determines an anisotropic charge-carrier mobility value for the organic molecule from the predicted intermolecular coupling property. The machine learning system then determines whether the anisotropic charge-carrier mobility value meets or exceeds a predetermined threshold anisotropic charge-carrier mobility value.

In embodiments, the machine learning system comprises at least a first machine learning model for determining a highest occupied molecular orbital (HOMO)-HOMO intermolecular electronic coupling property of the organic molecule and a second machine learning model for determining a lowest unoccupied molecular orbital (LUMO)-LUMO intermolecular electronic coupling property of the organic molecule. The machine learning system is in embodiments a graph neural network (GNN).

In embodiments, the molecular graph representations are three-dimensional noncovalent molecular dimer geometries derived from crystal structures of the at least two organic molecules. The molecular graph representations provide a position in Cartesian coordinates (x, y, z) and atomic number (Z) of all atoms in the noncovalent molecular dimer geometries.

In embodiments, the machine learning system determines a charge-carrier hopping probability (W) value for the organic molecules according to the formula:

$$W = \frac{{V}^{2}}{ℏ} \sqrt{\frac{π}{λ {k}_{B} T}} exp ( - \frac{λ}{4 {k}_{B} T} )$$

where V is an intermolecular electronic coupling, λ is a reorganization energy, T is a temperature, and k_{B} is a Boltzmann constant. The machine learning system further determines the anisotropic char e-carrier mobility value for the organic molecules according to the formula:

$${μ}_{ϕ} = \frac{e}{2 {k}_{B} T} {\sum }_{i} {W}_{i} {r}_{i}^{2} {P}_{i} {cos}^{2} {γ}_{i} {cos}^{2} ( {θ}_{i} - ϕ )$$

$${P}_{i} = \frac{{W}_{i}}{{\sum }_{i} {W}_{i}}$$

where i is a specific hopping path with a hopping distance of rᵢ, hopping rate Wᵢ, hopping probability Pᵢ. (θᵢ−ϕ) is the angle between the conducting channel and the hopping path, ϕ is the orientation of the conducting channel relative to the reference axis and γᵢ is the angle between the hopping paths and the reference plane. In embodiments, the predetermined threshold anisotropic charge-carrier mobility value is 1 cm²V⁻¹s⁻¹.

In another aspect, the present disclosure is directed to a machine learning system for modelling intermolecular electronic couplings of at least two organic molecules. The machine learning system comprises one or more non-transitory computer readable media storing computer-executable instructions and one or more processors configured to execute the computer-executable instructions to perform operations.

In embodiments, the operations comprise receiving and processing synthetically generated graph representations of the at least two organic molecules, wherein the graph representations are molecular graph representations in which each node corresponds to an atom of the organic molecule and each edge corresponds to a chemical bond between atoms of the organic molecules. Next, the machine learning system predicts an intermolecular coupling property (V) of the organic molecules from the molecular graph representations and, from the predicted intermolecular coupling property, determines an anisotropic charge-carrier mobility value for the organic molecules. The machine learning system then determines whether the anisotropic charge-carrier mobility value meets or exceeds a predetermined threshold anisotropic charge-carrier mobility value.

In embodiments, the machine learning system comprises at least a first machine learning model for determining a highest occupied molecular orbital (HOMO)-HOMO intermolecular electronic coupling property of the organic molecule and a second machine learning model for determining a lowest unoccupied molecular orbital (LUMO)-LUMO intermolecular electronic coupling property of the organic molecule. The machine learning system may be a graph neural network (GNN).

In embodiments, the molecular graph representations are three-dimensional noncovalent molecular dimer geometries derived from a crystal structure of the at least two organic molecules, wherein the molecular graph representations provide a position in Cartesian coordinates (x, y, z) and atomic number (Z) of all atoms in the noncovalent molecular dimer geometries.

In embodiments, the machine learning system determines a hopping probability (W) value for the at least two organic molecules according to the formula:

$$W = \frac{{V}^{2}}{ℏ} \sqrt{\frac{π}{λ {k}_{B} T}} exp ( - \frac{λ}{4 {k}_{B} T} )$$

where V is an intermolecular electronic coupling, A is a reorganization energy, T is a temperature, and k_{B} is a Boltzmann constant. The machine learning system may further determine the anisotropic charge-carrier mobility value for the at least two organic molecules according to the formula:

$${μ}_{ϕ} = \frac{e}{2 {k}_{B} T} {\sum }_{i} {W}_{i} {r}_{i}^{2} {P}_{i} {cos}^{2} {γ}_{i} {cos}^{2} ( {θ}_{i} - ϕ )$$

$${P}_{i} = \frac{{W}_{i}}{{\sum }_{i} {W}_{i}}$$

where i represents a specific hopping path with a hopping distance of rᵢ, hopping rate Wᵢ, hopping probability Pᵢ. (θᵢ−ϕ) is the angle between the conducting channel and the hopping path, ϕ is the orientation of the conducting channel relative to the reference axis and γᵢ is the angle between the hopping paths and the reference plane.

In embodiments, the intermolecular electronic coupling properties of the at least two organic molecules are modeled by the machine learning system for use in an optoelectronic application and the at least two organic molecules are selected for use in the optoelectronic application from a group of organic molecules determined by the machine learning system to have a charge-carrier mobility greater than 1 cm²V⁻¹s⁻¹ and an intermolecular electronic coupling anisotropy J_{avg}/J_{std}>1 for the intermolecular HOMO-HOMO intermolecular electronic coupling.

In yet another aspect, the present disclosure is directed to a method for training a machine learning-based system to select organic molecules suitable for use in an optoelectronic application. In embodiments, the optoelectronic application is an organic molecule-based semiconductor design.

The method includes configuring the machine learning-based system with at least a first machine learning model for determining a highest occupied molecular orbital (HOMO)-HOMO intermolecular electronic coupling property of the organic molecule and a second machine learning model for determining a lowest unoccupied molecular orbital (LUMO)-LUMO intermolecular electronic coupling property of the organic molecule.

The method further includes steps of inputting a plurality of synthetically generated graph representations of a plurality of organic molecules into the machine learning-based system. In embodiments, each graph representation of the plurality of synthetically generated graph representations is a molecular graph representation in which each node corresponds to an atom of an organic molecule of the plurality of organic molecules and each edge corresponds to a chemical bond between atoms of an organic molecule of the plurality of organic molecules. The machine learning system is configured to predict an intermolecular coupling property (V) of the plurality of organic molecules from the molecular graph representations and determines an anisotropic charge-carrier mobility value for the plurality of organic molecules from the predicted intermolecular coupling property. The machine learning-based system is further configure to select and output information relating to at least two organic molecules having a charge-carrier mobility greater than 1 cm²V⁻¹s⁻¹ and an intermolecular electronic coupling anisotropy J_{avg}/J_{std}>1 for the intermolecular HOMO-HOMO intermolecular electronic coupling. The machine learning-based system may in embodiments be a graph neural network (GNN).

In embodiments, the molecular graph representations are a three-dimensional noncovalent molecular dimer geometries derived from a crystal structure of the organic molecules, and provide a position in Cartesian coordinates (x, y, z) and atomic number (Z) of all atoms in the noncovalent molecular dimer geometries.

In embodiments, the machine learning system determines a hopping probability (W) value for the organic molecules according to the formula:

$$W = \frac{{V}^{2}}{ℏ} \sqrt{\frac{π}{λ {k}_{B} T}} exp ( - \frac{λ}{4 {k}_{B} T} )$$

where V is an intermolecular electronic coupling, λ is a reorganization energy, T is a temperature, and k_{B} is a Boltzmann constant. The machine learning system further determines the anisotropic charge-carrier mobility value for the organic molecules according to the formula:

$${μ}_{ϕ} = \frac{e}{2 {k}_{B} T} {\sum }_{i} {W}_{i} {r}_{i}^{2} {P}_{i} {cos}^{2} {γ}_{i} {cos}^{2} ( {θ}_{i} - ϕ )$$

$${P}_{i} = \frac{{W}_{i}}{{\sum }_{i} {W}_{i}}$$

where i represents a specific hopping path with a hopping distance of rᵢ, hopping rate Wᵢ, hopping probability Pᵢ. (θᵢ−ϕ) is the angle between the conducting channel and the hopping path, ϕ is the orientation of the conducting channel relative to the reference axis and γᵢ is the angle between the hopping paths and the reference plane.

It will be understood that various details of the presently disclosed subject matter can be changed without departing from the scope of the subject matter disclosed herein. Furthermore, the foregoing description is for the purpose of illustration only, and not for the purpose of limitation.

### BRIEF DESCRIPTION OF THE DRAWINGS

The presently disclosed subject matter will be better understood, and features, aspects and advantages other than those set forth above will become apparent when consideration is given to the following detailed description thereof. Such detailed description makes reference to the following drawings, wherein:

FIG. 1A shows a representative molecular dimer geometry consisting of two identical molecules at arbitrary relative separation and orientation. The atomic number (Z) and the Cartesian coordinates (x,y,z) features are labeled for the molecular dimer geometry and serve as input for the instant ML model.

FIG. 1B shows a message aggregation process for atom rₖ with message eₖ consisting of information from atoms sₖ and q.

FIG. 2 shows distributions for the OCELOT dimers v1 dataset. Histograms for the distribution of molecular size in terms of atoms (top left), kernel density estimate (KDE) for the center-of-mass distance between the dimers (top right), and the minimum intermolecular distance between the molecules (bottom left). The intermolecular HOMO-HOMO electronic coupling with the sign is shown in the KDE plot (bottom right) with the statistics for the absolute intermolecular HOMO-HOMO electronic couplings.

FIG. 3 depicts a scatter plot showing the correlation between the DFT estimated and ML predicted intermolecular HOMO-HOMO (top) and LUMO-LUMO (bottom) electronic couplings for the holdout test set consisting of 87,939 dimer configurations. The ML model was trained with the absolute values of intermolecular electronic couplings.

FIG. 4 shows scatter plots indicating the correlation between the DFT-derived and ML-predicted natural logarithm values of intermolecular HOMO-HOMO (left) and LUMO-LUMO (right) electronic couplings for the test set. (Top) The ML model was trained with the absolute values of intermolecular electronic couplings but plotted as a natural logarithm for comparison. (Bottom) The ML model was trained with a natural logarithm of the absolute value of the intermolecular electronic couplings.

FIG. 5 presents a histogram showing the average percent error for ML predictions of HOMO-HOMO coupling on the test dataset. A smaller bin is dedicated to the HOMO-HOMO coupling values between 0.001 and 0.010 eV as the MAE lies within this range, which causes large percent errors for the small couplings in this range.

FIG. 6 shows distributions with kernel density estimate (KDE) for the Spearman's rank correlation between the DFT and ML estimated HOMO-HOMO electronic couplings for the test set. The correlation is calculated for entries of crystal systems with three or more dimer configurations in the test dataset.

FIG. 7 illustrates variation of intermolecular HOMO-HOMO electronic coupling as a function of interplanar displacement (top) and displacement along the long-axis (bottom) for pentacene dimer. The direction of the red arrow between a dimer shows the displacement direction. DFT-derived data are in orange and ML predictions are in blue. The interplanar distance is set to 3.3 Å for the long-axis translation (bottom), and the long-axis displacement is set to 0 Å for the interplanar translation (top).

FIG. 8 illustrates variation of intermolecular HOMO-HOMO electronic couplings as a function of modifying the intermolecular distance between dimers of anthracene (top) and pentacene (bottom) along different directions indicated by the slab representation. DFT-derived data are in orange, and ML predictions are in blue. For the long-axis (center) and short-axis (right) translations, the vertical stacking distance is set to 3.3 Å. Note that the Y-axis labels for the plot in the center and right are the same as those on the left, i.e., HOMO-HOMO coupling (ev).

FIG. 9 illustrates variation of intermolecular HOMO-HOMO electronic couplings as a function of long-axis displacement between the face-to-face dimers of dinaphtho[2,3-b:2′,3′-f]thieno[3,2-b]thiophene (top), benzo[1,2-b:4,5-b′]dithiophene (middle, and [1]benzothieno[3,2-b][1]benzothiophene (bottom). DFT-derived data are in orange, and ML predictions are in blue. The vertical stacking distance is set to 4.0 Å.

FIG. 10 depicts the angular dependence of mobility (in cm²V⁻¹s⁻¹) in the ab plane for pentacene (left) and rubrene (right). Experimental estimates are in orange, and ML predictions are in blue. Experimental data for pentacene is from Ref.⁴⁸ with re-evaluations from Ref.⁴⁰ The experimental data for rubrene is from Ref.⁴⁹ (Bottom) The molecular structure of the top three crystals with high charge-carrier mobility as predicted by the ML pipeline. The ID corresponds to the Cambridge Structural Database (CSD) identifer.⁵⁰

### DETAILED DESCRIPTION

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

At a high level, the present disclosure is directed to use of a machine learning-based system for predicting intermolecular electronic coupling, for use in predicting suitability of organic molecules for various applications. In embodiments, the applications are optoelectronic applications. In embodiments, the optoelectronic applications are semiconductor designs. In embodiments, the semiconductor designs are organic molecule-based semiconductors.

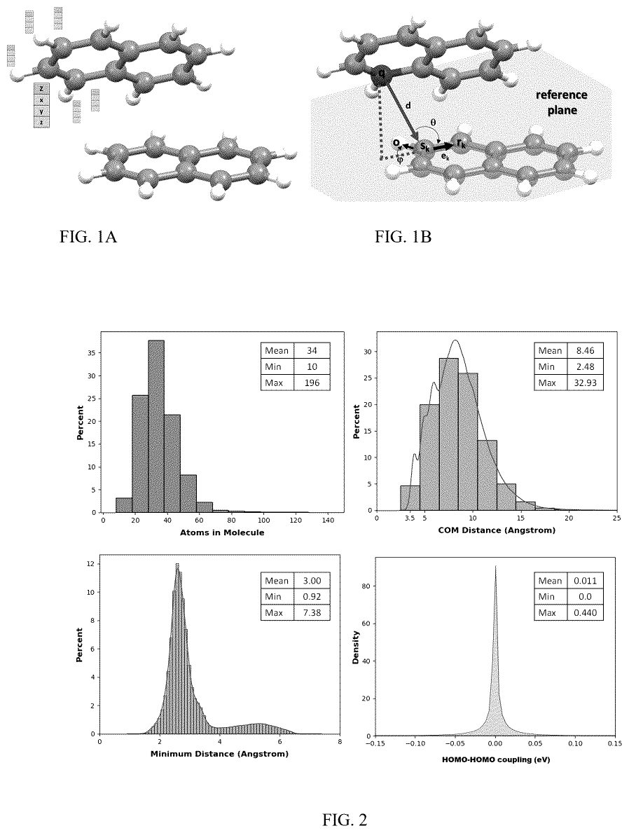

In one possible embodiment, the SphereNet model was used to predict intermolecular electronic coupling.⁵⁹ The input for training the ML model was the atomic Cartesian coordinates (x,y,z) and the corresponding atomic number (Z) in a molecular (noncovalent) dimer geometry, as shown in FIG. 1. During training, the Cartesian coordinates (x,y,z) were transformed to a 3D graph in spherical coordinates so that the corresponding tuple could specify the relative location of any atom in the dimer configuration (d, θ, ϕ). Any bond lengths can be defined by d, angles by 0, and torsions by 0, thus creating a rotation and translation invariant 3D graph. The translation and rotations referred to here are for the entire dimer configuration and not for individual molecules. For example, all the atoms in the dimer configuration are moved 5 Å in x; the relative position of the atoms does not change, which is modeled well by the 3D structure created in the training process. However, if the position of an atom changes, for instance, the atom labeled q in FIG. 1, the parameters d, θ, ϕ will change with respect to the origin labeled O.

During ML model training, a graph-based message-passing scheme was employed.⁶⁰ In the message-passing step, each atom in the dimer configuration accumulates information (message) from neighboring atoms. For instance, the atom labeled rₖ in FIG. 1 receives the message eₖ from atom sₖ. The message eₖ depends on the atoms surrounding s_{K}, except rₖ. The cutoff distance for determining the surrounding atoms was set to 5 Å. For each surrounding atom, say atom q, the message consists of spherical (\tilde{t}_{BF,lmn}), angular (ã_{SBF,ln}) and radial (\tilde{e}_{RBF,n}) part determined from the relative location (d, θ, ϕ).

$${\tilde{t}}_{BF , lmn} ( d , θ , ϕ ) = \sqrt{\frac{2}{{c}^{3} {j}_{l + 1}^{2} ( {z}_{ln} )}} {j}_{l} ( \frac{{z}_{ln}}{c} d ) {Y}_{l}^{m} ( θ , ϕ ) \qquad ( 3 )$$

$${\tilde{a}}_{SBF , ln} ( d , θ ) = \sqrt{\frac{2}{{c}^{3} {j}_{l + 1}^{2} ( {z}_{ln} )}} {j}_{l} ( \frac{{z}_{ln}}{c} d ) {Y}_{l}^{0} ( θ ) \qquad ( 4 )$$

$${\tilde{e}}_{RBF , n} ( d ) = \sqrt{\frac{2}{c}} ( \frac{sin ( \frac{n π}{c} d )}{d} ) \qquad ( 5 )$$

where c denotes the cutoff, zₗₙ is the n-th root of the Bessel function of order 1, jₗ(·) is a spherical Bessel function of order 1, Yₗ^{m} is a spherical harmonic function of degree m and order 1. The message eₖ is thus an aggregation of torsion, angular, and radial parts from all the neighboring atoms of s_{K}. Though the input to the ML model is a three-tuple (d, θ, ϕ), the Bessel functions provide more meaningful physical representation as the functional form shares similarity with the Schrödinger equation in spherical polar coordinates.⁶¹ Thus, the solutions to the Bessel functions were treated as the solutions to the Schrödinger equation.⁵⁹

### Methods

Dataset generation. In more detail, molecular (noncovalent) dimers from more than 25,000 crystal structures, both as solved via x-ray crystallography and minimized via DFT, were collected from the curated OCELOT database.³⁹ The screen_dimers function from the Hop module of the OCELOT API³⁹ was used to extract dimer geometries from the crystal structures. The extraction process involved identifying all the unique molecules in the unit cell of the crystal structure and searching for neighboring molecules within 5 Å of any atom in the unique molecule. The duplicates were removed by analyzing relative interplanar, long-axis, and short-axis displacements. The approach yielded various dimers for each structure depending on the number of molecules in the unit cell. Including DFT relaxed crystal structure for some entries doubles the number of dimer geometries. For instance, pentacene crystal (csd_PENCEN) with two unique molecules in the cell yielded 12 dimers for X-ray crystal structure and 12 dimers for DFT relaxed crystal structure, thus providing 24 dimer geometries for csd_PENCEN. The maximum total dimer geometry for a crystal structure in the dataset is 184. DFT single-point energy calculations were performed on the dimer geometries without further geometry optimization in Gaussian 16 Å.03⁵¹ at the PBE/6-31G(d,p) level of theory.⁵² The intermolecular electronic couplings were evaluated with the fragment molecular orbital (FMO) approach implemented in the OCELOT API.^{10, 13, 14,39} As noted, the FMO method used here accounts for polarization effects that arise from weak van der Waals intermolecular interactions. No additional corrections to the DFT functional were made to account for van der Waals interactions. The curated dataset contains 438,709 dimer geometries and corresponding intermolecular HOMO-HOMO and LUMO-LUMO electronic coupling values. This dataset, called OCELOT dimers v1, can be downloaded programmatically and from the OCELOT web user interface.

ML model. The Dive into Graphs implementation of SphereNet was used here.⁵³ Default hyperparameters were used, as tuning with Optuna version 2.1054 did not yield better performance. A 60:20:20 training: validation: test split of the dataset was used with mean square error (MSE) loss for training the ML model. The ML models were trained for 120 epochs, with a batch size of 32, Adam optimizer⁵⁵ with a learning rate of 0.0005, and a decay factor of 0.5 for 15 steps. Two ML models were trained—one for intermolecular HOMO-HOMO electronic coupling (for hole transport) and another for intermolecular LUMO-LUMO electronic coupling (for electron transport). ML model training was performed in PyTorch version 1.10 and used Cuda 11.4 for GPU acceleration on a single NVIDIA Tesla V100 GPU.^{56, 57} Each training epoch took 25 minutes.

Charge-carrier mobility. We implemented the formalism proposed by Goddard and coworkers to estimate charge-carrier mobility anisotropies.⁴⁰ The hopping rate W is evaluated using the semi-classical Marcus-Hush equation⁵⁸

$$W = \frac{{V}^{2}}{ℏ} \sqrt{\frac{π}{λ {k}_{B} T}} exp ( - \frac{λ}{4 {k}_{B} T} ) \qquad ( 6 )$$

where V is the intermolecular electronic coupling, λ is the reorganization energy, T is the temperature and k_{B} is the Boltzmann constant. The total reorganization constitutes the inner-sphere reorganization (intramolecular vibrational relaxations) and outer-sphere reorganization (polarization of the surrounding medium). Here, we assume the outer sphere reorganization energy contribution is of similar magnitude across the systems in the investigation; hence, we only consider changes in the inner-sphere reorganization energy when estimating the total reorganization energy. The values of V are obtained from the trained SphereNet model, and A is predicted by a previously published ML model.³⁷ The angular dependence of mobility is computed using the following equation.

$${μ}_{ϕ} = \frac{e}{2 {k}_{B} T} {\sum }_{i} {W}_{i} {r}_{i}^{2} {P}_{i} {cos}^{2} {γ}_{i} {cos}^{2} ( {θ}_{i} - ϕ ) \qquad ( 7 )$$

$${P}_{i} = \frac{{W}_{i}}{{\sum }_{i} {W}_{i}} \qquad ( 8 )$$

where i represents a specific hopping path with a hopping distance of rᵢ, hopping rate Wᵢ, hopping probability Pᵢ. (θᵢ−ϕ) is the angle between the conducting channel and the hopping path, ϕ is the orientation of the conducting channel relative to the reference axis and γᵢ is the angle between the hopping paths and the reference plane.

ML pipeline. The input to the pipeline is a crystallographic information file (CIF) from which the dimers and the largest, contiguous π-conjugated fragment of the molecule are extracted with OCELOT API. The reorganization energy is estimated for 2D SMILES representation of the largest, contiguous π-conjugated fragment using the fourth-generation pre-trained ML models from Bhat et al.³⁷ The intermolecular electronic coupling predictions obtained from the SphereNet model are then used to compute the anisotropic charge-carrier mobility along the various crystallographic planes. The temperature is set to 298 K. The ML pipeline is deployed on the OCELOT ML infrastructure.

### Results

The OCELOT dimer v1 dataset, which contains more than 438,000 dimers extracted from more than 25,000 (experimental and DFT-minimized) crystal structures in the OCELOT database, was used to train the ML model. Compared to a dataset generated through MD simulations, the OCELOT dimer v1 dataset may not capture all thermal molecular displacements. However, we hypothesized that the chemical diversity—the smallest molecular dimer in the dataset contains 20 atoms, while the largest has 392 atoms (see FIG. 2)—represented by the crystal structures makes the ML model trained on the OCELOT dataset more generalizable than previous ML models trained on more limited chemical spaces.^{28, 41} We note that while the signs of intermolecular electronic couplings are essential in determining the charge-carrier transport characteristics in molecular crystals through the transient localization theory model,^{22,42,43} initial efforts to train an ML model with the signs of the intermolecular electronic couplings yielded poor predictions. Hence, the model reported here was trained to predict absolute values of the intermolecular electronic couplings, which can be used as input in semi-classical evaluations of the electronic hopping rate constant in semi-classical Marcus-Hush theory. We used a 60:20:20 training:validation:test split of the dataset. Such a data split ensured that there were 125 unique crystal structures in the test set (see Table 1).

Table 1
Representative molecular structures for the 125 crystal systems unique to
the test set. The molecule on the left is present in the test set while the molecule on the right is
the closest match from the training set. The similarity score is computed using Tanimoto
similarity⁴ from RDKit. The correlation corresponds to Spearman's rank correlation between the
DFT and ML estimated HOMO-HOMO electronic couplings.

| Test Data Molecule | Train Data Molecule | Similarity | Correlation |
|---|---|---|---|
| 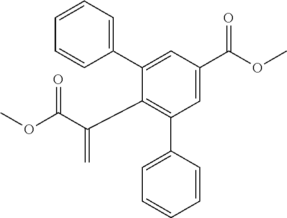 | 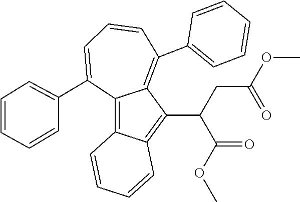 [SMILES: COC(=O)CC(C(=O)OC)c1c2c(-c3ccccc3)cccc(-c3ccccc3)c-2c2ccccc12] | 0.53 | 1.0 |
| 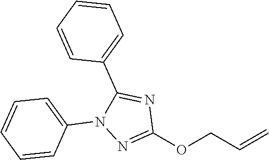 [SMILES: C=CCOc1nc(-c2ccccc2)n(-c2ccccc2)n1] | 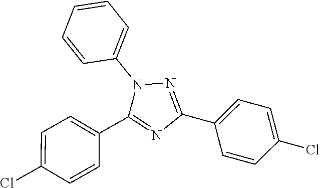 [SMILES: Clc1ccc(-c2nc(-c3ccc(Cl)cc3)n(-c3ccccc3)n2)cc1] | 0.61 | 0.5 |
| 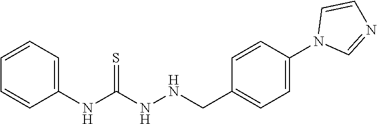 [SMILES: S=C(NNCc1ccc(-n2ccnc2)cc1)Nc1ccccc1] | 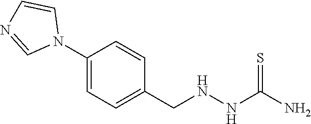 [SMILES: NC(=S)NNCc1ccc(-n2ccnc2)cc1] | 0.75 | 0.87 |
| 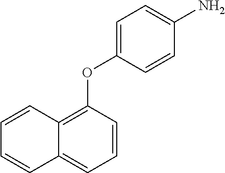 [SMILES: Nc1ccc(Oc2cccc3ccccc23)cc1] | 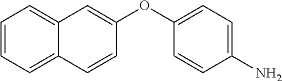 [SMILES: Nc1ccc(Oc2ccc3ccccc3c2)cc1] | 0.81 | 1.0 |
| 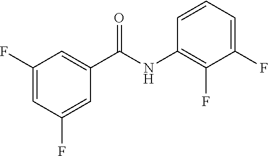 [SMILES: O=C(Nc1cccc(F)c1F)c1cc(F)cc(F)c1] | 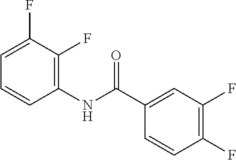 [SMILES: O=C(Nc1cccc(F)c1F)c1ccc(F)c(F)c1] | 0.91 | 0.98 |

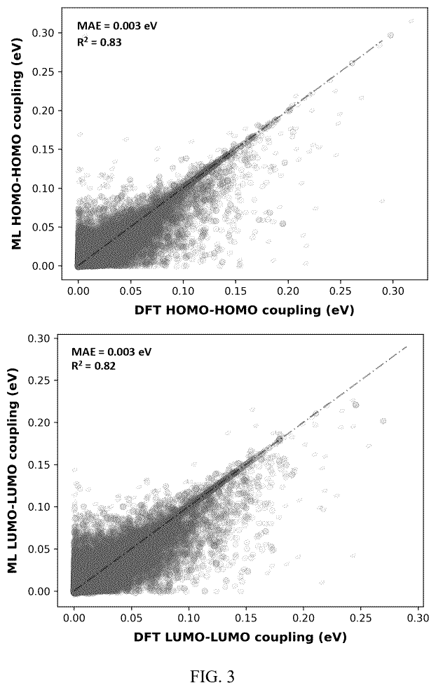

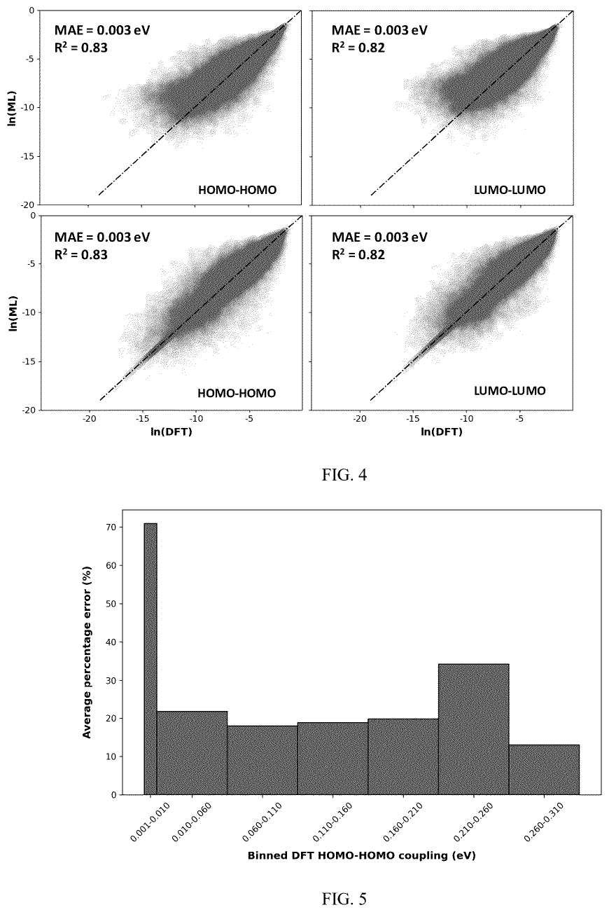

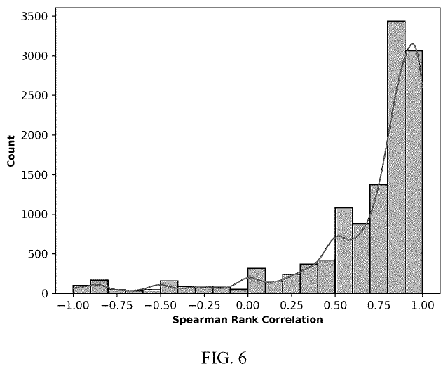

FIG. 3 demonstrates that the ML model produced reliable predictions of the intermolecular electronic couplings derived from DFT: The intermolecular HOMO-HOMO and LUMO-LUMO electronic couplings have mean absolute errors (MAE) of 3 meV and Pearson correlations (R²) of greater than 0.80. We implemented the natural logarithm of the absolute intermolecular electronic couplings for training to improve model performance, as demonstrated by Riderle et al.;²⁸ this training, however, did not significantly improve the performance but did lead to avoided saturation of values close to 0 meV (see FIG. 4). To gain insights into possible ML prediction errors, we analyzed the average percent error for the test dataset. As shown in FIG. 5, the average percent error is about 20%, suggesting that the ML model predictions are reliable over a large range of intermolecular electronic couplings. We note that, from the perspective of DFT evaluations of intermolecular electronic couplings, it is expected that the use of different DFT functionals and basis sets will lead to different coupling values;⁴⁴ hence, the ability to reproduce the trends of the intermolecular electronic couplings is more critical than reproducing the absolute values when making comparisons amongst different systems and models. We further determined the Spearman's rank correlation between the DFT-derived and ML-predicted HOMO-HOMO intermolecular electronic couplings; the results largely suggest a positive correlation (see FIG. 6), demonstrating that the ML model can predict well the trends in the DFT-derived intermolecular electronic couplings.

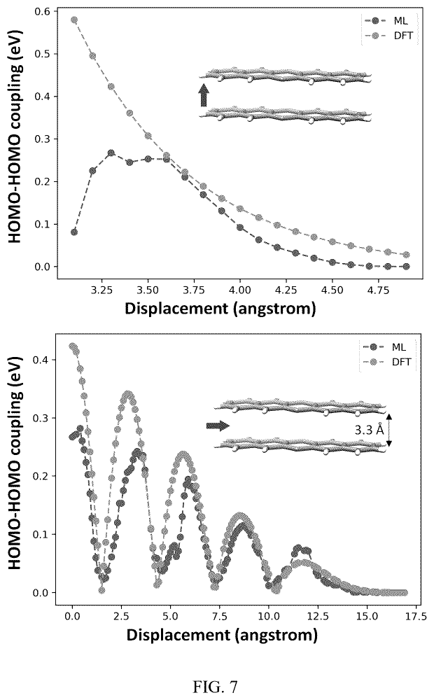

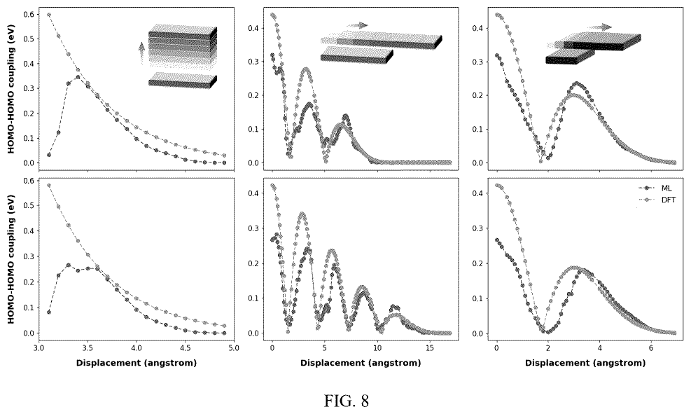

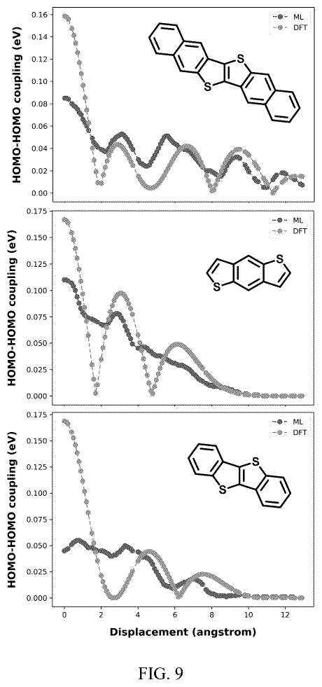

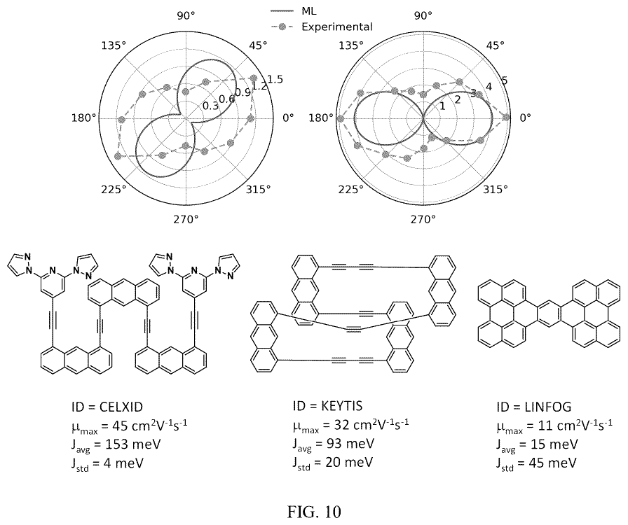

To further validate the observations, we analyzed the performance of the trained ML model to estimate the trends in intermolecular electronic couplings for pentacene. For the following discussion, we focus only on intermolecular HOMO-HOMO electronic couplings for a set of pentacene dimers with varied displacements—the dimer geometries were generated, using a Python code, by varying the interplanar, long-axis, and short-axis distances between the face-to-face packing of two molecules. Unlike previous ML models trained on over 10,000 molecular dimer geometries from MD snapshots for pentacene,²⁸ our dataset contains fewer than 400 pentacene dimer geometries from the 12 polymorphs and their DFT-relaxed geometries on which the ML model is trained. As shown in FIGS. 7-9, the ML model correctly predicted the trends of DFT-derived intermolecular electronic couplings, especially for interplanar separations in the range of 3.5-5.0 Å. We highlight, though, the discrepancies for interplanar separations less than 3.5 Å and the underestimation of large intermolecular electronic couplings. These discrepancies arise from sparse sampling of these regions in the datasets, as evident from FIG. 2, which is a consequence of the physics of the packing of π-conjugated organic molecules—there are very few crystal structures wherein the interplanar distance is less than 3.5 Å under standard experimental (temperature and pressure) conditions.

Though the ML model does generally underestimate the intermolecular electronic couplings with respect to the chosen DFT approach, predicting trends is sufficient for analyzing the relative values and trends among dimers in crystalline OSC and, thus, charge-carrier transport. With the ML-derived absolute intermolecular electronic coupling values, we next evaluate the charge-carrier transport anisotropies of crystalline OSC via semi-classical Marcus theory through the method proposed by Goddard and coworkers, which is insensitive to the sign of intermolecular electronic coupling (see equation 3).⁴⁰ We stress, of course, that this approach has significant limitations, as OSC charge-carrier mobilities can require descriptions from approaches that account for more delocalized charge-carrier wave functions, as described by, e.g., transient (de)localization or band transport mechanisms,^{22, 45-47} and non-local electron-phonon couplings. To forge a full ML pipeline to evaluate charge-carrier mobilities, the ML intermolecular electronic couplings described here were coupled to ML-derived estimates of the intramolecular reorganization energies, as previously described.³⁷ The performance of the ML pipeline was evaluated with pentacene and rubrene crystals, which show different angular anisotropies for charge-carrier transport.^{48,49} That the ML predictions tend to underestimate the intermolecular electronic couplings when compared to the DFT approach, this feature is propagated to estimated charge-carrier mobilities. The ML-evaluated angular dependencies in pentacene and rubrene crystals agree reasonably with the experiment, as shown in FIG. 10. While the angular dependence is accurately modeled for rubrene in FIG. 10, there is a discrepancy in the direction of the highest charge-carrier mobility for pentacene. We note that this discrepancy is inherent in the Marcus theory approach, as observed in the original article by Goddard and coworkers, wherein DFT calculations were used to evaluate these systems.⁴⁰

The ML pipeline estimated the charge-carrier mobility of a single crystal structure in less than one minute on a standard desktop (1-core Intel Xenon E3-1241, 4 GB RAM). We screened more than 110,000 crystal structures from the Cambridge Structural Database (2020.0.1 CSD release),⁵⁰ each of which consisted of one or more π-conjugated aromatic rings. We successfully screened approximately 60,000 of these structures for their charge-carrier mobilities; those structures that failed resulted from errors with parsing the crystal structure, missing hydrogen atoms, and the presence of metal atoms. The predictions indicate that highly π-conjugated molecules yield larger mobilities, as shown in FIG. 10, and only 372 structures presented an estimated maximum mobility (μₘₐₓ) greater than 1 cm²V⁻¹s⁻¹. Most systems show large intermolecular electronic coupling anisotropies, as evident from the average (J_{avg}) and standard deviation (J_{std}) intermolecular electronic couplings extracted (unique) dimers in the crystal structures. To focus on systems with low intermolecular electronic coupling anisotropies, we further filtered the down-selected 372 structures by identifying those with a ratio of J_{avg}/J_{std} greater than 1. Demonstrating the success of the ML-based screening approach, the resulting 40 down-selected structures (see Table 2) contain derivatives of well-known OSC, namely polyacenes. Some of these materials have reported charge-carrier mobilities (example: OKANUK; predicted=2.2 cmV⁻¹s⁻¹; experimental=1.12 cmV⁻¹s⁻¹), while others (example: ATOWUD, SECPUO, BISYAG, and KAJTEX) remain unexplored for charge-carrier transport applications. Of the 40 structures, the crystal structure of AQOSIJ is reported to have a phase transition above 125 K and hence would not be suitable for room temperature applications. Only six crystal structures (IPODEX, JEBNAG, SECPUO, NEVGUT, EDIHUV, KOYMES, DOGCEI) were measured at temperatures of 290 K or greater, while others were obtained in the range of 90K to 200K. We note, of course, that the charge-carrier transport analyses presented here are solely based on a hopping-based transport mechanism.

Table 2
40 potential candidates with i) μₘₐₓ over 1 cm²V⁻¹s⁻¹ and ii) low anisotropy,
i.e., J_{avg} / J_{std} is greater than 1.

| | | μₘₐₓ | J_{avg} | J_{std} |
|---|---|---|---|---|
| structure | CCDC ID | (cm²V⁻¹s⁻¹) | (eV) | (eV) |
| 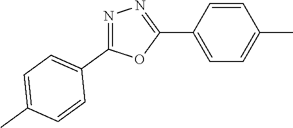 | IPODEX | 2.6 | 0.345 | 0.181 |
| 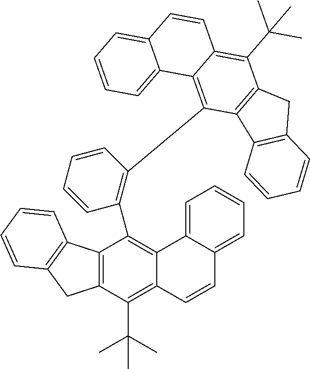 [SMILES: CC(C)(C)c1c2c(c(-c3ccccc3-c3c4c(c(C(C)(C)C)c5ccc6ccccc6c35)Cc3ccccc3-4)c3c1ccc1ccccc13)-c1ccccc1C2] | JEBNAG | 1.0 | 0.040 | 0.023 |
| 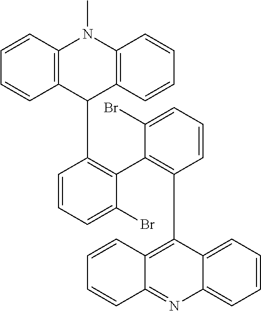 [SMILES: CN1c2ccccc2C(c2cccc(Br)c2-c2c(Br)cccc2-c2c3ccccc3nc3ccccc23)c2ccccc21] | SOSTUQ | 1.3 | 0.027 | 0.014 |
| 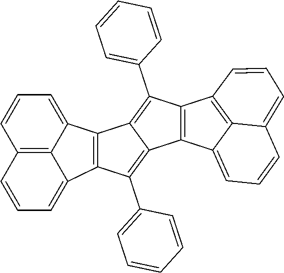 [SMILES: c1ccc(-c2c3c4cccc5cccc(c=3c3c(-c6ccccc6)c6c7cccc8cccc(c=6c23)c87)c54)cc1] | OVILOW | 1.6 | 0.029 | 0.026 |
| 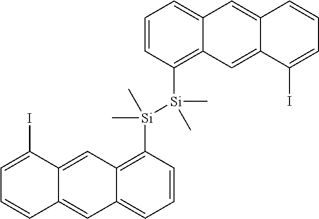 [SMILES: C[Si](C)(c1cccc2cc3cccc(I)c3cc12)[Si](C)(C)c1cccc2cc3cccc(I)c3cc12] | INIWAE | 8.9 | 0.127 | 0.019 |
| 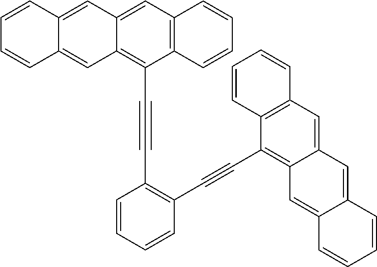 [SMILES: C(#Cc1c2ccccc2cc2cc3ccccc3cc12)c1ccccc1C#Cc1c2ccccc2cc2cc3ccccc3cc12] | KAJTEX | 9.4 | 0.084 | 0.011 |
| 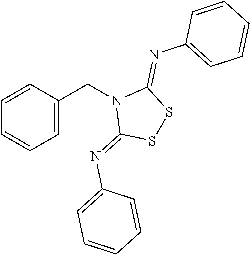 [SMILES: c1ccc(Cn2/c(=N/c3ccccc3)ss/c2=N\c2ccccc2)cc1] | BPIMTZ | 1.0 | 0.037 | 0.028 |
| 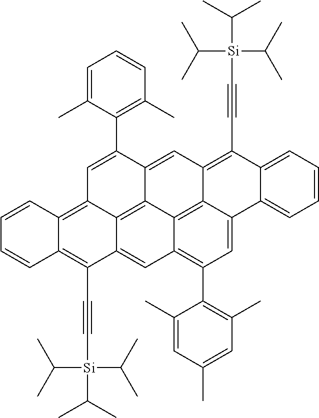 [SMILES: Cc1cc(C)c(-c2cc3c4ccccc4c(C#C[Si](C(C)C)(C(C)C)C(C)C)c4cc5c(-c6c(C)cccc6C)cc6c7ccccc7c(C#C[Si](C(C)C)(C(C)C)C(C)C)c7cc2c(c43)c5c76)c(C)c1] | SECPUO | 1.2 | 0.018 | 0.017 |
| 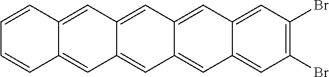 [SMILES: Brc1cc2cc3cc4cc5ccccc5cc4cc3cc2cc1Br] | OKANOE | 1.7 | 0.053 | 0.037 |
| 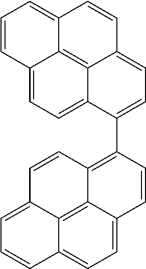 [SMILES: c1cc2ccc3ccc(-c4ccc5ccc6cccc7ccc4c5c67)c4ccc(c1)c2c34] | CAFVEN | 1.2 | 0.030 | 0.026 |
| 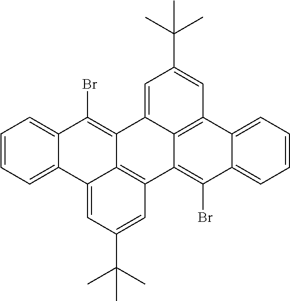 [SMILES: CC(C)(C)c1cc2c3ccccc3c(Br)c3c4cc(C(C)(C)C)cc5c6ccccc6c(Br)c(c(c1)c23)c54] | BISYAG | 2.8 | 0.028 | 0.028 |
| 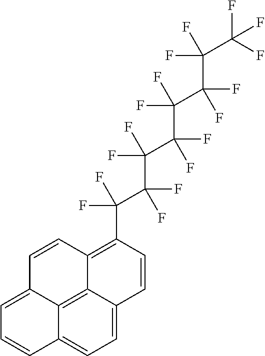 [SMILES: FC(F)(F)C(F)(F)C(F)(F)C(F)(F)C(F)(F)C(F)(F)C(F)(F)C(F)(F)c1ccc2ccc3cccc4ccc1c2c34] | MABZEW | 1.4 | 0.042 | 0.042 |
| 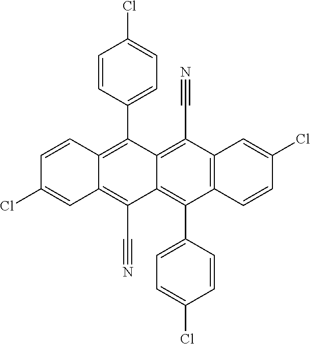 [SMILES: N#Cc1c2cc(Cl)ccc2c(-c2ccc(Cl)cc2)c2c(C#N)c3cc(Cl)ccc3c(-c3ccc(Cl)cc3)c12] | COGBIL | 1.6 | 0.035 | 0.031 |
| 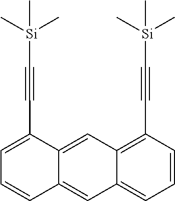 [SMILES: C[Si](C)(C)C#Cc1cccc2cc3cccc(C#C[Si](C)(C)C)c3cc12] | QOKKIM | 1.3 | 0.023 | 0.023 |
| 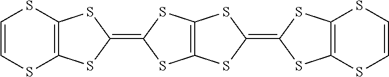 [SMILES: C1=CSC2=C(S1)SC(=C1SC3=C(S1)SC(=C1SC4=C(SC=CS4)S1)S3)S2] | KOYMES | 2.4 | 0.053 | 0.047 |
| 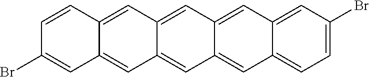 [SMILES: Brc1ccc2cc3cc4cc5cc(Br)ccc5cc4cc3cc2c1] | OKANUK | 2.2 | 0.064 | 0.042 |
| 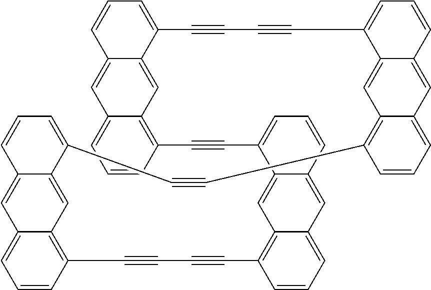 [SMILES: C1#Cc2cccc3cc4cccc(c4cc23)C#Cc2cccc3cc4cccc(c4cc23)C#CC#Cc2cccc3cc4cccc(c4cc23)C#Cc2cccc3cc4cccc(c4cc23)C#C1] | KEYTIS | 32.5 | 0.094 | 0.020 |
| 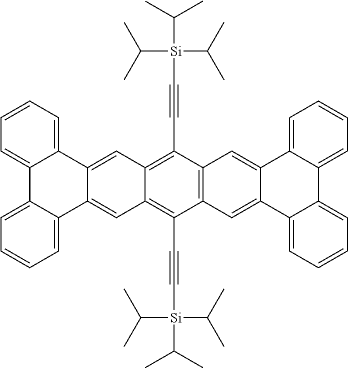 [SMILES: CC(C)[Si](C#Cc1c2cc3c4ccccc4c4ccccc4c3cc2c(C#C[Si](C(C)C)(C(C)C)C(C)C)c2cc3c4ccccc4c4ccccc4c3cc12)(C(C)C)C(C)C] | QEWHAE01 | 1.7 | 0.027 | 0.023 |
| 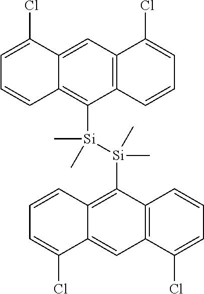 [SMILES: C[Si](C)(c1c2cccc(Cl)c2cc2c(Cl)cccc12)[Si](C)(C)c1c2cccc(Cl)c2cc2c(Cl)cccc12] | HIFXIG | 1.4 | 0.062 | 0.004 |
| 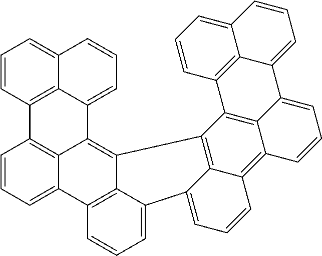 [SMILES: c1cc2cccc3c2c(c1)c1cccc2c4cccc5c6cccc7c8cccc9c%10cccc%11cccc(c%11%10)c(c98)c(c76)c(c45)c3c12] | NEVGUT | 1.7 | 0.017 | 0.014 |
| 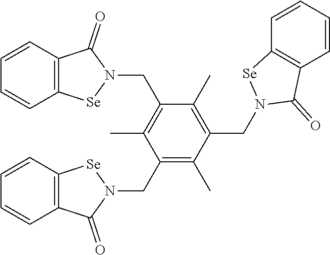 [SMILES: Cc1c(Cn2[se]c3ccccc3c2=O)c(C)c(Cn2[se]c3ccccc3c2=O)c(C)c1Cn1[se]c2ccccc2c1=O] | EDIHUV | 3.3 | 0.125 | 0.015 |
| 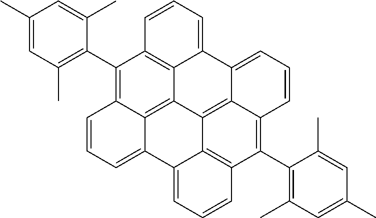 | CEXQUT | 1.5 | 0.027 | 0.026 |
| 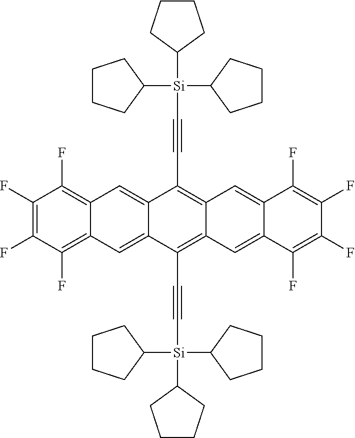 [SMILES: Fc1c(F)c(F)c2cc3c(C#C[Si](C4CCCC4)(C4CCCC4)C4CCCC4)c4cc5c(F)c(F)c(F)c(F)c5cc4c(C#C[Si](C4CCCC4)(C4CCCC4)C4CCCC4)c3cc2c1F] | OZUWUD | 4.2 | 0.046 | 0.040 |
| 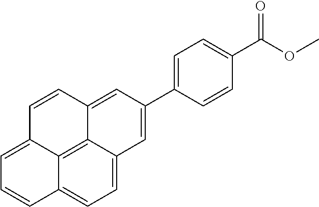 [SMILES: COC(=O)c1ccc(-c2cc3ccc4cccc5ccc(c2)c3c45)cc1] | CARLEO | 1.1 | 0.032 | 0.027 |
| 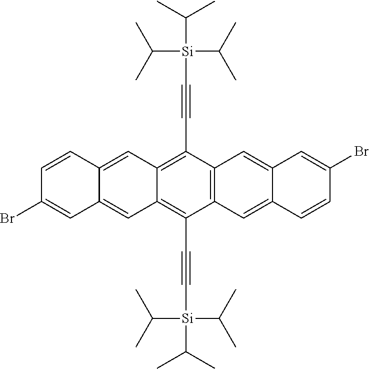 [SMILES: CC(C)[Si](C#Cc1c2cc3ccc(Br)cc3cc2c(C#C[Si](C(C)C)(C(C)C)C(C)C)c2cc3ccc(Br)cc3cc12)(C(C)C)C(C)C] | AQOSIJ | 6.3 | 0.049 | 0.048 |
| 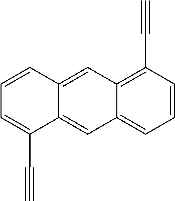 [SMILES: C#Cc1cccc2cc3c(C#C)cccc3cc12] | QOKKOS | 1.1 | 0.020 | 0.014 |
| 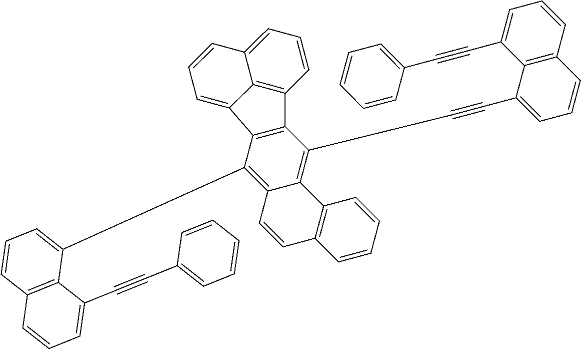 [SMILES: C(#Cc1cccc2cccc(C#Cc3c4c(c(-c5cccc6cccc(C#Cc7ccccc7)c56)c5ccc6ccccc6c35)-c3cccc5cccc-4c35)c12)c1ccccc1] | TAPRUZ | 1.4 | 0.036 | 0.005 |
| 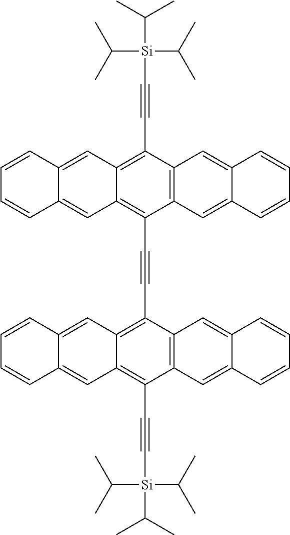 [SMILES: CC(C)[Si](C#Cc1c2cc3ccccc3cc2c(C#Cc2c3cc4ccccc4cc3c(C#C[Si](C(C)C)(C(C)C)C(C)C)c3cc4ccccc4cc23)c2cc3ccccc3cc12)(C(C)C)C(C)C] | VIMQAM | 4.2 | 0.043 | 0.036 |
| 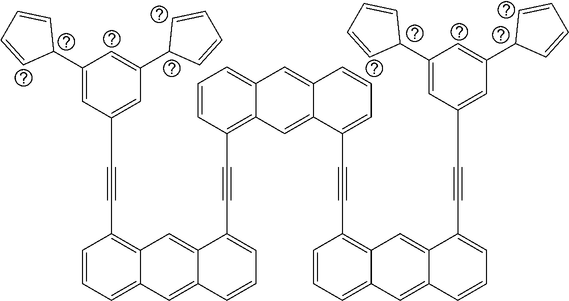 [SMILES: C(#Cc1cccc2cc3cccc(C#Cc4cccc5cc6cccc(C#Cc7cccc8cc9cccc(C#Cc%10cc(C%11C=CC=C%11)cc(C%11C=CC=C%11)c%10)c9cc78)c6cc45)c3cc12)c1cc(C2C=CC=C2)cc(C2C=CC=C2)c1] | CELXID | 45.1 | 0.153 | 0.005 |
| 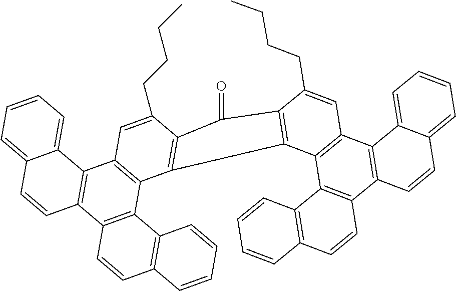 [SMILES: CCCCc1cc2c(c3c1C(=O)c1c(CCCC)cc4c(c1-3)c1c3ccccc3ccc1c1ccc3ccccc3c14)c1c3ccccc3ccc1c1ccc3ccccc3c12] | BAQCEE | 1.2 | 0.010 | 0.007 |
| 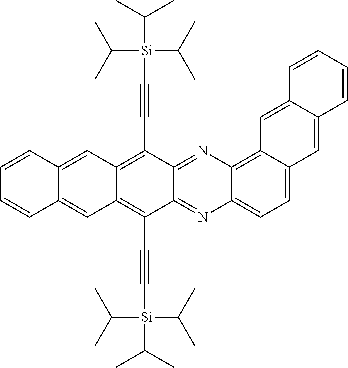 [SMILES: CC(C)[Si](C#Cc1c2cc3ccccc3cc2c(C#C[Si](C(C)C)(C(C)C)C(C)C)c2nc3c(ccc4cc5ccccc5cc43)nc12)(C(C)C)C(C)C] | OVIWIB | 1.9 | 0.022 | 0.017 |
| 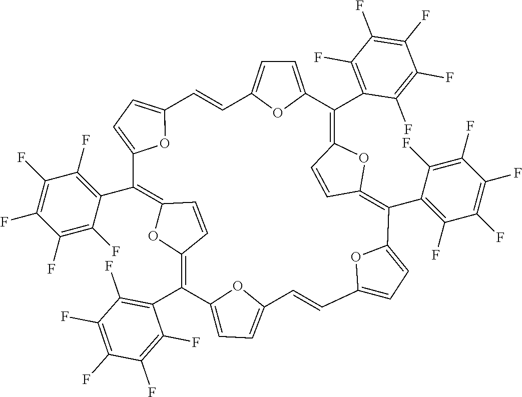 [SMILES: Fc1c(F)c(F)c(/C2=C3C=C/C(=C(/c4c(F)c(F)c(F)c(F)c4F)C4=CC=C(/C=C/c5ccc(o5)/C(c5c(F)c(F)c(F)c(F)c5F)=C5C=C/C(=C(/c6c(F)c(F)c(F)c(F)c6F)C6=CC=C(/C=C/C7=CC=C2O7)O6)O\5)O4)O/3)c(F)c1F] | ROFVIT | 1.1 | 0.089 | 0.012 |
| 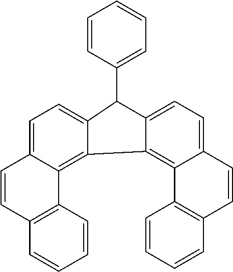 [SMILES: c1ccc(C2c3ccc4ccc5ccccc5c4c3-c3c2ccc2ccc4ccccc4c32)cc1] | ISILON | 1.0 | 0.016 | 0.015 |
| 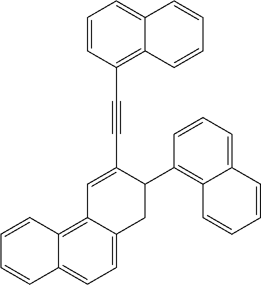 [SMILES: C(#Cc1cccc2ccccc12)C1=Cc2c(ccc3ccccc23)CC1c1cccc2ccccc12] | DOGCEI | 1.3 | 0.021 | 0.017 |
| 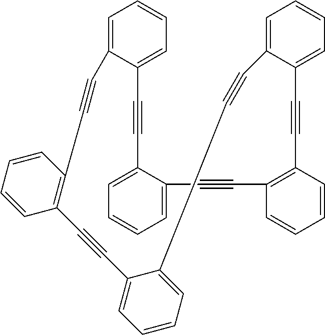 [SMILES: C1#Cc2ccccc2C#Cc2ccccc2C#Cc2ccccc2C#Cc2ccccc2C#Cc2ccccc2C#Cc2ccccc21] | WEXWIF | 2.5 | 0.045 | 0.009 |
| 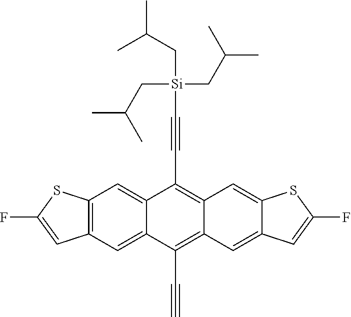 [SMILES: C#Cc1c2cc3cc(F)sc3cc2c(C#C[Si](CC(C)C)(CC(C)C)CC(C)C)c2cc3sc(F)cc3cc12] | ATOWUD | 1.1 | 0.041 | 0.038 |
| 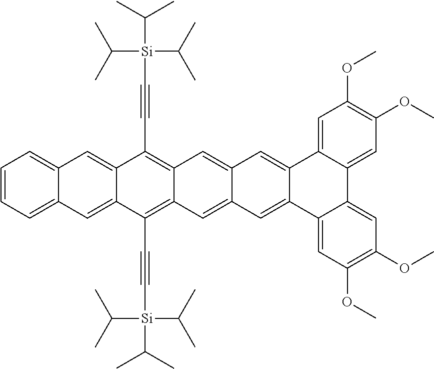 [SMILES: COc1cc2c3cc(OC)c(OC)cc3c3cc4cc5c(C#C[Si](C(C)C)(C(C)C)C(C)C)c6cc7ccccc7cc6c(C#C[Si](C(C)C)(C(C)C)C(C)C)c5cc4cc3c2cc1OC] | RESTAN | 1.5 | 0.035 | 0.028 |
| 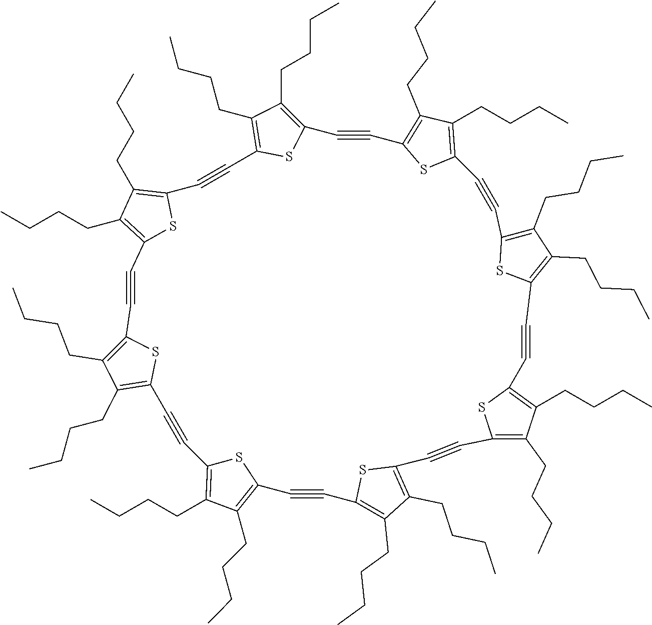 [SMILES: CCCCC1=C2C#CC3=C(CCCC)C(CCCC)=C(C#CC4=C(CCCC)C(CCCC)=C(C#CC5=C(CCCC)C(CCCC)=C(C#CC6=C(CCCC)C(CCCC)=C(C#CC7=C(CCCC)C(CCCC)=C(C#CC8=C(CCCC)C(CCCC)=C(C#CC9=C(CCCC)C(CCCC)=C(C#CC(=C1CCCC)S2)S9)S8)S7)S6)S5)S4)S3] | LULWEW | 1.4 | 0.024 | 0.022 |
| 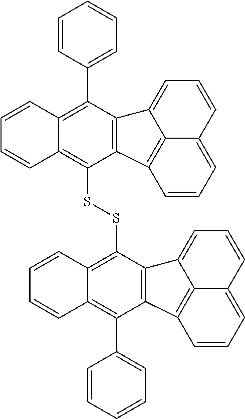 [SMILES: c1ccc(-c2c3c(c(SSc4c5c(c(-c6ccccc6)c6ccccc46)-c4cccc6cccc-5c46)c4ccccc24)-c2cccc4cccc-3c24)cc1] | WICCUG | 1.1 | 0.042 | 0.015 |
| 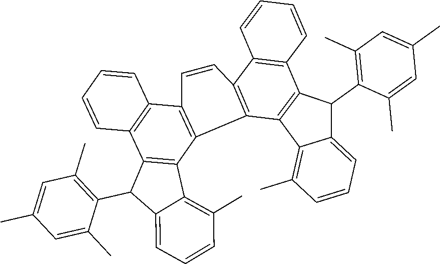 | WIKGOP | 1.2 | 0.03 | 0.01 |

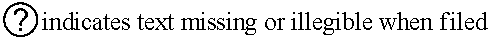

In summary, we report a ML model to predict intermolecular electronic couplings from 3D geometries of crystals comprised of π-conjugated organic molecules and an associated ML pipeline to evaluate charge-carrier mobility anisotropies in crystalline molecular OSC. Trained on the diverse 438,000 OCELOT dimers v1 dataset, we demonstrate that the MPNN-based model is reliable in predicting the trends in intermolecular electronic couplings. The ML model is transferable to molecules of different sizes, and we anticipate that this strategy can be deployed over a vast chemical space. Adopting the semi-classical Marcus theory formulation, an ML-based pipeline was created and deployed to evaluate charge-carrier mobilities in organic crystals. More than 60,000 crystalline molecular materials were screened for their charge-carrier transport properties via the ML pipeline, and 40 potential candidates with charge-carrier mobilities greater than 1 cm²V⁻¹s⁻¹ and low anisotropies in the intermolecular HOMO-HOMO electronic couplings were identified. Importantly, several of the down-selected molecular crystals are well-known and experimentally verified systems in terms of their charge-carrier transport properties, demonstrating the validity of the proof-of-concept screening approach. With rapid predictions of intermolecular electronic couplings and charge-carrier mobilities, these ML models can be used in fast analyses of charge-carrier transport that are coupled with MD simulation or kinetic Monte Carlo approaches and eventually in a complete materials discovery suite that involves searches of molecular space, crystal structure prediction, and material property prediction.

While the terms used herein are believed to be well understood by those of ordinary skill in the art, certain definitions are set forth to facilitate explanation of the presently disclosed subject matter.

Unless defined otherwise, all technical and scientific terms used herein have the same meaning as is commonly understood by one of skill in the art to which the invention(s) belong.

Any and all patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Unless otherwise indicated, all numbers expressing quantities or values are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about” is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

All publications, patents, and patent applications mentioned in this specification are herein incorporated by reference to the same extent as if each individual publication, patent, or patent application was specifically and individually indicated to be incorporated by reference.

It will be understood that various details of the presently disclosed subject matter can be changed without departing from the scope of the subject matter disclosed herein. Furthermore, the foregoing description is for the purpose of illustration only, and not for the purpose of limitation. Obvious modifications and variations are possible in light of the above teachings. All such modifications and variations are within the scope of the appended claims when interpreted in accordance with the breadth to which they are fairly, legally and equitably entitled.

### REFERENCES

ADDIN EN.REFLIST (1) Thorley, K. J.; Risko, C. Mapping the configuration dependence of electronic coupling in organic semiconductors. J. Mater. Chem. C Mater 2016, 4 (17), 3825-3832. DOI: 10.1039/c5tc03765d
(2) Zhang, Z.; Jiang, L.; Cheng, C.; Zhen, Y.; Zhao, G.; Geng, H.; Yi, Y.; Li, L.; Dong, H.; Shuai, Z.; et al. The Impact of Interlayer Electronic Coupling on Charge Transport in Organic Semiconductors: A Case Study on Titanylphthalocyanine Single Crystals. Angew. Chem. Int. Ed. 2016, 55 (17), 5206-5209. DOI: 10.1002/anie.201601065
(3) Brédas, J. L.; Calbert, J. P.; Da Silva Filho, D. A.; Cornil, J. Organic semiconductors: A theoretical characterization of the basic parameters governing charge transport. PNAS 2002, 99 (9), 5804-5809. DOI: 10.1073/pnas.092143399
(4) Coropceanu, V.; Cornil, J.; Da Silva Filho, D. A.; Olivier, Y.; Silbey, R.; Brddas, J.-L. Charge Transport in Organic Semiconductors. Chem. Rev. 2007, 107 (4), 926-952. DOI: 10.1021/cr050140x
(5) Bhat, V.; Callaway, C. P.; Risko, C. Computational Approaches for Organic Semiconductors: From Chemical and Physical Understanding to Predicting New Materials. Chem. Rev. 2023, 123 (12), 7498-7547. DOI: 10.1021/acs.chemrev.2c00704
(6) Sutton, C.; Marshall, M. S.; Sherrill, C. D.; Risko, C.; Brddas, J.-L. Rubrene: The Interplay between Intramolecular and Intermolecular Interactions Determines the Planarization of Its Tetracene Core in the Solid State. J. Am. Chem. Soc. 2015, 137 (27), 8775-8782. DOI: 10.1021/jacs.5b04066
(7) Kubas, A.; Gajdos, F.; Heck, A.; Oberhofer, H.; Elstner, M.; Blumberger, J. Electronic couplings for molecular charge transfer: benchmarking CDFT, FODFT and FODFTB against high-level ab initio calculations. II. Phys. Chem. Chem. Phys. 2015, 17 (22), 14342-14354. DOI: 10.1039/c4cp04749d
(8) Lemaur, V.; Da Silva Filho, D. A.; Coropceanu, V.; Lehmann, M.; Geerts, Y.; Piris, J.; Debije, M. G.; Van De Craats, A. M.; Senthilkumar, K.; Siebbeles, L. D. A.; et al. Charge Transport Properties in Discotic Liquid Crystals: A Quantum-Chemical Insight into Structure-Property Relationships. J. Am. Chem. Soc. 2004, 126 (10), 3271-3279. DOI: 10.1021/ja0390956
(9) Holstein, T. Studies of polaron motion: Part II. The “small” polaron. Ann. Phys. (NY) 1959, 8 (3), 343-389. DOI: 10.1016/0003-4916(59)90003-X
(10) Valeev, E. F.; Coropceanu, V.; Da Silva Filho, D. A.; Salman, S.; Brddas, J.-L. Effect of Electronic Polarization on Charge-Transport Parameters in Molecular Organic Semiconductors. J. Am. Chem. Soc. 2006, 128 (30), 9882-9886. DOI: 10.1021/ja061827h
(11) Cave, R. J.; Newton, M. D. Calculation of electronic coupling matrix elements for ground and excited state electron transfer reactions: Comparison of the generalized Mulliken-Hush and block diagonalization methods. J. Chem. Phys. 1997, 106 (22), 9213-9226. DOI: 10.1063/1.474023
(12) Hutchison, G. R.; Ratner, M. A.; Marks, T. J. Intermolecular Charge Transfer between Heterocyclic Oligomers. Effects of Heteroatom and Molecular Packing on Hopping Transport in Organic Semiconductors. J. Am. Chem. Soc. 2005, 127 (48), 16866-16881. DOI: 10.1021/ja0533996
(13) Newman, C. R.; Frisbie, C. D.; Da Silva Filho, D. A.; Brddas, J.-L.; Ewbank, P. C.; Mann, K. R. Introduction to Organic Thin Film Transistors and Design of n-Channel Organic Semiconductors. Chem. Mater. 2004, 16 (23), 4436-4451. DOI: 10.1021/cm049391x
(14) Senthilkumar, K.; Grozema, F. C.; Guerra, C. F.; Bickelhaupt, F. M.; Lewis, F. D.; Berlin, Y. A.; Ratner, M. A.; Siebbeles, L. D. A. Absolute Rates of Hole Transfer in DNA. J. Am. Chem. Soc. 2005, 127 (42), 14894-14903. DOI: 10.1021/ja054257e
(15) Kobori, T.; Sodeyama, K.; Otsuka, T.; Tateyama, Y.; Tsuneyuki, S. Trimer effects in fragment molecular orbital-linear combination of molecular orbitals calculation of one-electron orbitals for biomolecules. J. Chem. Phys. 2013, 139 (9). DOI: 10.1063/1.4818599
(16) Giannini, S.; Blumberger, J. Charge Transport in Organic Semiconductors: The Perspective from Nonadiabatic Molecular Dynamics. Acc. Chem. Res. 2022. DOI: 10.1021/acs.accounts.1c00675
(17) Troisi, A.; Cheung, D. L.; Andrienko, D. Charge Transport in Semiconductors with Multiscale Conformational Dynamics. Phys. Rev. Lett. 2009, 102 (11). DOI: 10.1103/physrevlett.102.116602
(18) Brddas, J.-L.; Beljonne, D.; Coropceanu, V.; Cornil, J. Charge-Transfer and Energy-Transfer Processes in rr-Conjugated Oligomers and Polymers: A Molecular Picture. Chem. Rev. 2004, 104 (11), 4971-5004. DOI: 10.1021/cr040084k
(19) Cornil, J.; Beljonne, D.; Calbert, J.-P.; Brddas, J.-L. Interchain Interactions in Organic π-Conjugated Materials: Impact on Electronic Structure, Optical Response, and Charge Transport. Adv. Mater. 2001, 13 (14), 1053-1067. DOI: 10.1002/1521-4095(200107)13:14<1053::AID-ADMA1053>3.0.CO;2-7
(20) Kubai, T.; Elstner, M. Efficient algorithms for the simulation of non-adiabatic electron transfer in complex molecular systems: application to DNA. Phys. Chem. Chem. Phys. 2013, 15 (16), 5794. DOI: 10.1039/c3cp44619k
(21) Kubai, T.; Elstner, M. A hybrid approach to simulation of electron transfer in complex molecular systems. Journal of The Royal Society Interface 2013, 10 (87), 20130415. DOI: 10.1098/rsif.2013.0415
(22) Fratini, S.; Mayou, D.; Ciuchi, S. The Transient Localization Scenario for Charge Transport in Crystalline Organic Materials. Adv. Funct. Mater. 2016, 26 (14), 2292-2315. DOI: 10.1002/adfm.201502386
(23) Ciuchi, S.; Fratini, S.; Mayou, D. Transient localization in crystalline organic semiconductors. Phys. Rev. B. 2011, 83 (8). DOI: 10.1103/physrevb.83.081202
(24) Lederer, J.; Kaiser, W.; Mattoni, A.; Gagliardi, A. Machine Learning-Based Charge Transport Computation for Pentacene. Adv. Theory Simul. 2019, 2 (2), 1800136. DOI: 10.1002/adts.201800136
(25) Bai, X.; Guo, X.; Wang, L. Machine Learning Approach to Calculate Electronic Couplings between Quasi-diabatic Molecular Orbitals: The Case of DNA. J. Phys. Chem. Lett. 2021, 12 (42), 10457-10464. DOI: 10.1021/acs.jpclett.1c03053
(26) Reiser, P.; Konrad, M.; Fediai, A.; Léon, S.; Wenzel, W.; Friederich, P. Analyzing Dynamical Disorder for Charge Transport in Organic Semiconductors via Machine Learning. J. Chem. Theory. Comput. 2021, 17 (6), 3750-3759. DOI: 10.1021/acs.jctc.1c00191
(27) Wang, C.-I.; Joanito, I.; Lan, C.-F.; Hsu, C.-P. Artificial neural networks for predicting charge transfer coupling. J. Chem. Phys. 2020, 153 (21), 214113. DOI: 10.1063/5.0023697
(28) Rinderle, M.; Kaiser, W.; Mattoni, A.; Gagliardi, A. Machine-Learned Charge Transfer Integrals for Multiscale Simulations in Organic Thin Films. Journal of Physical Chemistry C 2020, 124 (32), 17733-17743. DOI: 10.1021/acs.jpcc.0c04355
(29) Bag, S.; Aggarwal, A.; Maiti, P. K. Machine Learning Prediction of Electronic Coupling between the Guanine Bases of DNA. J. Phys. Chem. A 2020, 124 (38), 7658-7664. DOI: 10.1021/acs.jpca.0c04368
(30) Tan, T.; Wang, D. Machine learning based charge mobility prediction for organic semiconductors. J. Chem. Phys. 2023, 158 (9), 094102. DOI: 10.1063/5.0134379
(31) Cignoni, E.; Cupellini, L.; Mennucci, B. Machine Learning Exciton Hamiltonians in Light-Harvesting Complexes. J. Chem. Theory. Comput. 2023, 19 (3), 965-977. DOI: 10.1021/acs.jctc.2c01044
(32) Farahvash, A.; Lee, C.-K.; Sun, Q.; Shi, L.; Willard, A. P. Machine learning Frenkel Hamiltonian parameters to accelerate simulations of exciton dynamics. J. Chem. Phys. 2020, 153 (7), 074111. DOI: 10.1063/5.0016009
(33) Schafer, C.; Fojt, J.; Lindgren, E.; Erhart, P. Machine Learning for Polaritonic Chemistry: Accessing Chemical Kinetics. J. Am. Chem. Soc. 2024. DOI: 10.1021/jacs.3c12829
(34) Rupp, M.; Tkatchenko, A.; Muller, K.-R.; Von Lilienfeld, O. A. Fast and Accurate Modeling of Molecular Atomization Energies with Machine Learning. Phys. Rev. Lett. 2012, 108 (5). DOI: 10.1103/physrevlett.108.058301
(35) Rinderle, M.; Gagliardi, A. Machine Learning & multiscale simulations: toward fast screening of organic semiconductor materials. In 2021 International Conference on Numerical Simulation of Optoelectronic Devices (NUSOD), 2021-09-13, 2021; IEEE. DOI: 10.1109/nusod52207.2021.9541414.
(36) Sanchez-Lengeling, B.; Reif, E.; Pearce, A.; Wiltschko, A. B. A gentle introduction to graph neural networks. Distill 2021, 6 (9), e33. DOI: 10.23915/distill.00033
(37) Bhat, V.; Sornberger, P.; Pokuri, B. S. S.; Duke, R.; Ganapathysubramanian, B.; Risko, C. Electronic, redox, and optical property prediction of organic 71-conjugated molecules through a hierarchy of machine learning approaches. Chem. Sci. 2023, 14 (1), 203-213. DOI: 10.1039/d2sc04676h
(38) Liu, Y.; Wang, L.; Liu, M.; Zhang, X.; Oztekin, B.; Ji, S. Spherical Message Passing for 3D Graph Networks. ArXiv 2021. DOI: 10.48550/arXiv.2102.05013
(39) Ai, Q.; Bhat, V.; Ryno, S. M.; Jarolimek, K.; Sornberger, P.; Smith, A.; Haley, M. M.; Anthony, J. E.; Risko, C. OCELOT: An infrastructure for data-driven research to discover and design crystalline organic semiconductors. J. Chem. Phys. 2021, 154 (17), 174705. DOI: 10.1063/5.0048714
(40) Wen, S.-H.; Li, A.; Song, J.; Deng, W.-Q.; Han, K.-L.; Goddard, W. A. First-Principles Investigation of Anistropic Hole Mobilities in Organic Semiconductors. J. Phys. Chem. B. 2009, 113 (26), 8813-8819. DOI: 10.1021/jp900512s
(41) Wang, C.-I.; Braza, M. K. E.; Claudio, G. C.; Nellas, R. B.; Hsu, C.-P. Machine Learning for Predicting Electron Transfer Coupling. J. Phys. Chem. A. 2019, 123 (36), 7792-7802. DOI: 10.1021/acs.jpca.9b04256
(42) Troisi, A. Charge transport in high mobility molecular semiconductors: classical models and new theories. Chem. Soc. Rev. 2011, 40 (5), 2347. DOI: 10.1039/cOcs00198h
(43) Fratini, S.; Ciuchi, S.; Mayou, D.; De Laissardiere, G. T.; Troisi, A. A map of high-mobility molecular semiconductors. Nat. Mater. 2017, 16 (10), 998-1002. DOI: 10.1038/nmat4970
(44) Fonari, A.; Sutton, C.; Brédas, J.-L.; Coropceanu, V. Impact of exact exchange in the description of the electronic structure of organic charge-transfer molecular crystals. Phys. Rev. B. 2014, 90 (16). DOI: 10.1103/physrevb.90.165205
(45) Giannini, S.; Peng, W.-T.; Cupellini, L.; Padula, D.; Carof, A.; Blumberger, J. Exciton transport in molecular organic semiconductors boosted by transient quantum delocalization. Nat. Commun. 2022, 13 (1). DOI: 10.1038/s41467-022-30308-5
(46) Fratini, S.; Ciuchi, S. Dynamical localization corrections to band transport. Physical Review Research 2020, 2 (1). DOI: 10.1103/physrevresearch.2.013001
(47) Leblanc, O. H. Band Structure and Transport of Holes and Electrons in Anthracene. J. Chem. Phys. 1961, 35 (4), 1275-1280. DOI: 10.1063/1.1732038
(48) Lee, J. Y.; Roth, S.; Park, Y. W. Anisotropic field effect mobility in single crystal pentacene. Appl. Phys. Lett. 2006, 88 (25), 252106. DOI: 10.1063/1.2216400
(49) Reese, C.; Bao, Z. High-Resolution Measurement of the Anisotropy of Charge Transport in Single Crystals. Adv. Mater. 2007, 19 (24), 4535-4538. DOI: 10.1002/adma.200701139
(50) Groom, C. R.; Bruno, I. J.; Lightfoot, M. P.; Ward, S. C. The Cambridge structural database. Acta. Crystallogr. B. Struct. Sci. Cryst. Eng. Mater. 2016. DOI: 10.1107/S2052520616003954
(51) Frisch, G. W.; Schlegel, H. B.; Scuseria, G. E.; Robb, M. A.; Cheeseman, J. R.; Scalmani, G.; Barone, V.; Petersson, G. A.; Nakatsuji, H.; Li, X.; et al. Gaussian 16, Rev. A.03. 2016.
(52) Perdew, J. P.; Burke, K.; Ernzerhof, M. Generalized gradient approximation made simple. Phys. Rev. Lett. 1996, 77 (18), 3865-3868. DOI: 10.1103/PhysRevLett.77.3865
(53) Liu, M.; Luo, Y.; Wang, L.; Xie, Y.; Yuan, H.; Gui, S.; Xu, Z.; Yu, H.; Zhang, J.; Liu, Y.; et al. DIG: A Turnkey Library for Diving into Graph Deep Learning Research. J. Mach. Learn. Res. 2021, 22, 240:241-240:249.
(54) Takuya, A.; Shotaro, S.; Toshihiko, Y.; Takeru, O.; Masanori, K. Optuna: A Next-generation Hyperparameter Optimization Framework In Proceedings of the 25th ACM SIGKDD International Conference on Knowledge Discovery &amp; Data Mining, Association for Computing Machinery: Anchorage, AK, USA, 2019; pp 2623-2631.
(55) Kingma, D. P.; Ba, J. Adam: A method for stochastic optimization. arXiv 2014. DOI: 10.48550/arXiv.1412.6980
(56) Paszke, A.; Gross, S.; Massa, F.; Lerer, A.; Bradbury, J.; Chanan, G.; Killeen, T.; Lin, Z.; Gimelshein, N.; Antiga, L.; et al. PyTorch: An Imperative Style, High-Performance Deep Learning Library. In NeurIPS, 2019.
(57) Nickolls, J.; Buck, I.; Garland, M.; Skadron, K. Scalable Parallel Programming with CUDA. Queue 2008, 6 (2), 40-53. DOI: 10.1145/1365490.1365500
(58) Marcus, R. A. Chemical and Electrochemical Electron-Transfer Theory. Annu. Rev. Phys. Chem. 1964, 15 (1), 155-196. DOI: 10.1146/annurev.pc.15.100164.001103
ADDIN EN.REFLIST (59) Liu, Y.; Wang, L.; Liu, M.; Zhang, X.; Oztekin, B.; Ji, S. Spherical Message Passing for 3D Graph Networks. ArXiv 2021. DOI: 10.48550/arXiv.2102.05013
(60) Sanchez-Lengeling, B.; Reif, E.; Pearce, A.; Wiltschko, A. B. A gentle introduction to graph neural networks. Distill 2021, 6 (9), e33. DOI: 10.23915/distill.00033
(61) Jensen, F. Introduction to Computational Chemistry; 2017. DOI: 10.1007/s00214-013-1372-6.
(62) Bajusz, D.; Räcz, A.; Héberger, K. Why is Tanimoto index an appropriate choice for fingerprint-based similarity calculations?J. Cheminform. 2015, 7 (1), 20. DOI: 10.1186/s13321-015-0069-3

What is claimed:

1. A machine learning-based method for modelling the intermolecular electronic couplings of organic molecules, comprising:
inputting into a machine learning system synthetically generated graph representations of at least two organic molecules, wherein the graph representations are molecular graph representations in which each node corresponds to an atom of the organic molecules and each edge corresponds to a chemical bond between atoms of the organic molecules;
by the machine learning system, predicting an intermolecular coupling property (V) between the at least two organic molecules from the molecular graph representations;
by the machine learning system, determining an anisotropic charge-carrier mobility value for the organic molecule from the predicted intermolecular coupling property; and
by the machine learning system, determining whether the anisotropic charge-carrier mobility value meets or exceeds a predetermined threshold anisotropic charge-carrier mobility value.

2. The method of claim 1, wherein the machine learning system comprises at least a first machine learning model for determining a highest occupied molecular orbital (HOMO)-HOMO intermolecular electronic coupling property of the organic molecule and a second machine learning model for determining a lowest unoccupied molecular orbital (LUMO)-LUMO intermolecular electronic coupling property of the organic molecule.

3. The method of claim 1, wherein the machine learning system is a graph neural network (GNN).

4. The method of claim 1, wherein the molecular graph representations are three-dimensional noncovalent molecular dimer geometries derived from crystal structures of the at least two organic molecules.

5. The method of claim 4, wherein the molecular graph representations provide a position in Cartesian coordinates (x, y, z) and atomic number (Z) of all atoms in the noncovalent molecular dimer geometries.

6. The method of claim 1, wherein the machine learning system determines:
a charge-carrier hopping probability (W) value for the organic molecules according to the formula:
$$W = \frac{{V}^{2}}{ℏ} \sqrt{\frac{π}{λ {k}_{B} T}} exp ( - \frac{λ}{4 {k}_{B} T} )$$
where V is an intermolecular electronic coupling, λ is a reorganization energy, T is a temperature, and k_{B} is a Boltzmann constant; and
the anisotropic charge-carrier mobility value for the organic molecules according to the formula:
$${μ}_{ϕ} = \frac{e}{2 {k}_{B} T} {\sum }_{i} {W}_{i} {r}_{i}^{2} {P}_{i} {cos}^{2} {γ}_{i} {cos}^{2} ( {θ}_{i} - ϕ )$$
$${P}_{i} = \frac{{W}_{i}}{{\sum }_{i} {W}_{i}}$$
where i is a specific hopping path with a hopping distance of rᵢ, hopping rate Wᵢ, hopping probability Pᵢ. (θᵢ−ϕ) is the angle between the conducting channel and the hopping path, ϕ is the orientation of the conducting channel relative to the reference axis and γᵢ is the angle between the hopping paths and the reference plane.

7. The method of claim 6, wherein the predetermined threshold anisotropic charge-carrier mobility value is 1 cm²V⁻¹s⁻¹.

8. A machine learning system for modelling intermolecular electronic couplings of at least two organic molecules, comprising:
one or more non-transitory computer readable media storing computer-executable instructions; and
one or more processors configured to execute the computer-executable instructions to perform operations comprising:
receiving and processing synthetically generated graph representations of the at least two organic molecules, wherein the graph representations are molecular graph representations in which each node corresponds to an atom of the organic molecule and each edge corresponds to a chemical bond between atoms of the organic molecules;
predicting an intermolecular coupling property (V) of the organic molecules from the molecular graph representations;
from the predicted intermolecular coupling property, determining an anisotropic charge-carrier mobility value for the organic molecules; and
determining whether the anisotropic charge-carrier mobility value meets or exceeds a predetermined threshold anisotropic charge-carrier mobility value.

9. The system of claim 8, wherein the machine learning system comprises at least a first machine learning model for determining a highest occupied molecular orbital (HOMO)-HOMO intermolecular electronic coupling property of the organic molecule and a second machine learning model for determining a lowest unoccupied molecular orbital (LUMO)-LUMO intermolecular electronic coupling property of the organic molecule.

10. The system of claim 8, wherein the machine learning system is a graph neural network (GNN).

11. The system of claim 8, wherein the molecular graph representations are three-dimensional noncovalent molecular dimer geometries derived from a crystal structure of the at least two organic molecules.

12. The system of claim 11, wherein the molecular graph representations provide a position in Cartesian coordinates (x, y, z) and atomic number (Z) of all atoms in the noncovalent molecular dimer geometries.

13. The system of claim 8, wherein the machine learning system determines:
a hopping probability (W) value for the at least two organic molecules according to the formula:
$$W = \frac{{V}^{2}}{ℏ} \sqrt{\frac{π}{λ {k}_{B} T}} exp ( - \frac{λ}{4 {k}_{B} T} )$$
where V is an intermolecular electronic coupling, λ is a reorganization energy, T is a temperature, and k_{B} is a Boltzmann constant; and
the anisotropic charge-carrier mobility value for the at least two organic molecules according to the formula:
$${μ}_{ϕ} = \frac{e}{2 {k}_{B} T} {\sum }_{i} {W}_{i} {r}_{i}^{2} {P}_{i} {cos}^{2} {γ}_{i} {cos}^{2} ( {θ}_{i} - ϕ )$$
$${P}_{i} = \frac{{W}_{i}}{{\sum }_{i} {W}_{i}}$$
where i represents a specific hopping path with a hopping distance of rᵢ, hopping rate Wᵢ, hopping probability Pᵢ. (θᵢ−ϕ) is the angle between the conducting channel and the hopping path, ϕ is the orientation of the conducting channel relative to the reference axis and γⱼ is the angle between the hopping paths and the reference plane.

14. The system of claim 13, wherein the intermolecular electronic coupling properties of the at least two organic molecules are modeled for use in an optoelectronic application and the at least two organic molecules are selected for use in the optoelectronic application from a group of organic molecules determined by the machine learning system to have a charge-carrier mobility greater than 1 cm²V⁻¹s⁻¹ and an intermolecular electronic coupling anisotropy J_{avg}/J_{std}>1 for the intermolecular HOMO-HOMO intermolecular electronic coupling.

15. A method for training a machine learning-based system to select organic molecules suitable for use in an optoelectronic application, comprising:
configuring the machine learning-based system with at least a first machine learning model for determining a highest occupied molecular orbital (HOMO)-HOMO intermolecular electronic coupling property of the organic molecule and a second machine learning model for determining a lowest unoccupied molecular orbital (LUMO)-LUMO intermolecular electronic coupling property of the organic molecule;
inputting into the machine learning-based system a plurality of synthetically generated graph representations of a plurality of organic molecules, wherein each graph representation of the plurality of synthetically generated graph representations is a molecular graph representation in which each node corresponds to an atom of an organic molecule of the plurality of organic molecules and each edge corresponds to a chemical bond between atoms of an organic molecule of the plurality of organic molecules;
by the machine learning system, predicting an intermolecular coupling property (V) of the plurality of organic molecules from the molecular graph representations;
by the machine learning-based system, determining an anisotropic charge-carrier mobility value for the plurality of organic molecules from the predicted intermolecular coupling property; and
by the machine learning-based system, selecting and outputting from the plurality of organic molecules at least two organic molecules having a charge-carrier mobility greater than 1 cm²V⁻¹s⁻¹ and an intermolecular electronic coupling anisotropy J_{avg}/J_{std}>1 for the intermolecular HOMO-HOMO intermolecular electronic coupling.

16. The method of claim 15, wherein the machine learning-based system is a graph neural network (GNN).

17. The method of claim 15, wherein the molecular graph representations are a three-dimensional noncovalent molecular dimer geometries derived from a crystal structure of the organic molecules.

18. The method of claim 17, wherein the molecular graph representations provide a position in Cartesian coordinates (x, y, z) and atomic number (Z) of all atoms in the noncovalent molecular dimer geometries.

19. The method of claim 15, wherein the machine learning system determines:
a hopping probability (W) value for the organic molecules according to the formula:
$$W = \frac{{V}^{2}}{ℏ} \sqrt{\frac{π}{λ {k}_{B} T}} exp ( - \frac{λ}{4 {k}_{B} T} )$$
where V is an intermolecular electronic coupling, λ is a reorganization energy, T is a temperature, and k_{B} is a Boltzmann constant; and
the anisotropic charge-carrier mobility value for the organic molecules according to the formula:
$${μ}_{ϕ} = \frac{e}{2 {k}_{B} T} {\sum }_{i} {W}_{i} {r}_{i}^{2} {P}_{i} {cos}^{2} {γ}_{i} {cos}^{2} ( {θ}_{i} - ϕ )$$
$${P}_{i} = \frac{{W}_{i}}{{\sum }_{i} {W}_{i}}$$
where i represents a specific hopping path with a hopping distance of rᵢ, hopping rate Wᵢ, hopping probability Pᵢ. (θᵢ−ϕ) is the angle between the conducting channel and the hopping path, ϕ is the orientation of the conducting channel relative to the reference axis and γᵢ is the angle between the hopping paths and the reference plane.

20. The method of claim 15, wherein the optoelectronic application is an organic molecule-based semiconductor design.
